(19) United States
(12) Patent Application Publication
(10) Pub. No.: US 2023/0355907 A1
(43) Pub. Date: Nov. 9, 2023

(54) NASAL CANNULA INTERFACE

(71) Applicant: Fisher & Paykel Healthcare Limited, Auckland (NZ)

(72) Inventors: Callum James Thomas Spence, Auckland (NZ); Michael Paul Ronayne, Auckland (NZ); Monika Baumann, Auckland (NZ); Rory Alexander Monro, Auckland (NZ); Andrew James Hilliard, Auckland (NZ); Georgia Gadsby, Auckland (NZ)

(21) Appl. No.: 18/044,756

(22) PCT Filed: Sep. 10, 2021
(86) PCT No.: PCT/NZ2021/050160

§ 371 Date: Mar. 9, 2023

Related U.S. Application Data
(60) Provisional application No. 63/077,105, filed on Sep. 11, 2020.

(51) Int. Cl. A61M 16/06; A61M 16/16; A61M 16/08; A61M 16/00

(52) U.S. Cl. CPC A61M 16/0666; A61M 16/16; A61M 16/0683; A61M 16/0875; A61M 16/022

(57) ABSTRACT

A nasal cannula interface is provided for a respiratory support system configured to receive a breathable gases flow, the nasal cannula interface comprising: a. an inlet to receive the gases flow; b. at least one nasal prong configured to receive the gases flow from the inlet, and to be received in, and to deliver the gases flow to, a nare of the patient. The nasal cannula interface may comprise one or more structural features that are configured to help manage, avoid and/or reduce generation of aerosols by the patient during breathing and/or whilst breathing gases from a respiratory support apparatus.

### INCORPORATIONS BY REFERENCE

International Application No. PCT/NZ2017/050119, titled “Thermistor Flow Sensor Having Multiple Temperature Points”, filed on Sep. 13, 2017, International Application No. PCT/NZ2016/050193, titled “Flow Path Sensing for Respiratory support Apparatus”, filed on Dec. 2, 2016, International Application No. PCT/IB2016/053761, titled “Breathing Assistance Apparatus”, filed on Jun. 24, 2016, and PCT/NZ2016/050101 filed on Jun. 27 2016, titled “Exhalation Port”, International Application No. PCT/NZ2013/000166 filed Sep. 9 2013, International application PCT/IB2015/054585 (WO2015/193833) published 23 Dec. 2015, PCT/NZ2013/000113 (WO2014003579), filed 25 Jun. 2013 are hereby incorporated by reference in their entireties. This application claims priority from U.S. provisional patent application 63/077,105 filed 11 Sep. 2020, the entire contents of which is hereby incorporated by reference in their entireties.

### FIELD OF THE DISCLOSURE

The present disclosure relates to a nasal cannula interface configured for use with a respiratory support apparatus comprising a flow generator configured to generate a gases flow for inspiration by a patient.

### BACKGROUND

Respiratory support apparatuses are used in various environments such as hospital, medical facilities, residential care, or home environments to deliver a gases flow to users or patients. Respiratory support apparatus comprise a flow generator to generate a gases flow, and/or can receive a gases flow from another gases source such as a hospital oxygen wall source for example.

The gases flow is delivered to the patient via an inspiratory conduit and a nasal cannula interface, the nasal cannula interface being mounted on the head of the patient, for example using headgear.

One example of a respiratory support apparatus is a high flow respiratory support apparatus, to deliver a high flow of heated, and typically humidified, gases to a patient via a nasal cannula. Such a respiratory support apparatus can sometimes be configured to selectively deliver more than one type of respiratory therapy, for example CPAP/Bilevel and High Flow therapies. A typical high flow apparatus comprises a (NHF therapy). The nasal cannula may comprise one or more prongs configured to be received in the nares of the patient. The prongs may be configured not to seal against the nares, such that gases, including exhalation gases, can leak around the prongs.

It has been found that undesirable matter, such as pathogens, microbes, other disease carrying matter and/or undesirable substances/contaminants can be present in the expiratory gases generated by a patient when exhaling. In some cases, such matter comprises aerosols. Aerosols can carry undesirable matter in the form of contaminants that include biological agents, chemical substances (e.g. medicaments) or other particles that its undesirable for those in close proximity with the patient or person to come into contact with. For example, in a patient suffering from infectious diseases e.g. COVID 19, SARS, MERS, or any other infectious diseases, the aerosols can carry these diseases. The exhaled gases, including any aerosols, can present a risk of carrying and spreading these diseases.

Both NHF and CPAP/Bilevel circuits, as currently in use, typically have gas flow exiting from the circuit, that contain or mix with gas exhaled from the patient. These gas flows are vented from the circuit and thus can present a risk of contamination by carrying the undesirable matter. Preventing or reducing the dispersion of aerosols may reduce transmission of this undesirable matter. In one example, this could reduce transmission of one or more infectious diseases. However, the source of overflow of contaminated gases differs in NHF and CPAP/bilevel, but in both treatment devices carries the same consequence. In NHF, the flow delivered to the patient is relatively high (for example greater than 20 l/min) and in excess of that inhaled during each breath. During exhalation, this high flow entrains the patient's exhalation through the nose around a non-sealing nasal prong. In CPAP/bilevel, the excess flow delivered to the patient need only be enough to maintain pressure in the (sealed) mask and circuit, but a leak must be inserted in the circuit to allow exhaled gases from the patient to exit prior to each inhalation, to prevent rebreathing of CO2.

Non-invasive (NIV) therapies, such as Nasal High Flow (NHF), typically involve uni-directional gas flow though the respiratory apparatus towards the patient. Therefore, aerosols generated upstream of the patient are not likely to be infectious. In many cases non-invasive therapies do not include provision for capture of exhaled gases. For example in NHF, the exhaled gases typically leak from around the nasal cannula interface, into atmosphere. These exhaled gases can include aerosols generated by the patient which could carry undesirable matter to which caregivers and others might be exposed.

### SUMMARY OF DISCLOSURE

The present disclosure relates to a nasal cannula interface comprising one or more structural features that are configured to help manage, avoid and/or reduce generation of aerosols by the patient during breathing and/or whilst breathing gases from a respiratory support apparatus.

The present disclosure relates to managing, avoiding, and/or reducing generation of aerosols that are, or are more likely to become, airborne. In some cases, such aerosols can be less than about 140 microns.

In accordance with this disclosure the nasal cannula interface may comprise a body or additional component that is configured to alter or control the inspiratory and/or expiratory flow path through or around the nasal cannula interface.

In accordance with this disclosure the nasal cannula interface may be provided with integral features that are configured to alter or control the inspiratory and/or expiratory flow path through or around the nasal cannula interface.

In accordance with this disclosure the nasal cannula interface may be provided with features configured to alter or control the characteristics of the inspiratory and/or expiratory gases flow through and/or around the nasal cannula interface.

In accordance with this disclosure the nasal cannula interface may be a non-invasive ventilation (NIV) interface.

In accordance with this disclosure the nasal cannula interface may comprise one or more prongs configured to be received in the nare(s) of the patient, where one or more of the prongs have one or more features configured to alter to control the inspiratory and/or expiratory flow path through or around the prong(s) and/or to alter or control the characteristics of the inspiratory and/or expiratory gases flow through and/or around the prong(s).

The one or more prongs may be non-sealing prongs, sealing prongs, or a combination of non-sealing and sealing prongs.

In accordance with this disclosure the or each nasal prong may comprise a prong inlet opening to receive a breathable gases flow, a prong outlet opening to deliver the breathable gases flow to the patient's nares, and a flow path through the prong, between the inlet opening and outlet opening.

In accordance with this disclosure any one or more of the following features of at least one prong of the nasal cannula assembly may be configured to reduce aerosolisation:
a. length of prongs between the inlet and outlet openings;
b. curvature of prongs between the inlet and outlet openings;
c. cross sectional size of the flow path between the inlet and outlet openings;
d. cross sectional shape of the flow path between the inlet and outlet openings;
e. shape of inlet opening;
f. shape of outlet opening;
g. size of inlet opening;
h. size of outlet opening;
i. relative size of inlet and outlet openings;
j. surface contouring of the flow path between the inlet and outlet openings;
k. surface contouring of the exterior surface(s) of the prong(s);

In accordance with this disclosure the or each nasal prong may comprise a plurality of openings and a plurality of flow paths through the prong. In some embodiments one flow path is an inspiratory flow path, and the other flow path is an expiratory flow path.

In accordance with this disclosure at least one prong of the nasal cannula assembly may be configured such that:
a. a predetermined part of the nasal cannula assembly contacts and/or seals with, with the patient's nare(s);
b. a predetermined part of the nasal cannula assembly does not contact and/or seal with the patient's nare(s);
c. a predetermined part of the nasal cannula assembly is spaced from the patient's nares.

In accordance with this disclosure the size, shape, length and/or configuration of an inspiratory flow path through the prong(s) of the nasal cannula is configured to control aerosolization.

In accordance with this disclosure the size, shape, length and/or configuration of an expiratory flow path around the prong(s) of the nasal cannula is configured to control aerosolization.

In accordance with this disclosure the size, shape, length and/or configuration of an expiratory flow path through the prong(s) of the nasal cannula is configured to control aerosolization.

In accordance with this disclosure the nasal cannula interface may comprise material in the inspiratory and/or expiratory flow path through or around the nasal cannula interface, the material comprising:
a. a porous material;
b. a wicking material;
c. a diffuser material.

In accordance with this disclosure the material is provided on, in or adjacent the prong(s) and/or on a manifold from which the prong(s) depend.

In accordance with this disclosure the nasal cannula interface is configured in particular to mitigate the airborne infection risk and therefore the generation of aerosols smaller than ˜120 microns.

In accordance with this disclosure there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient;
wherein the nasal prong defines a plurality of gas flow paths extending through the prong.

One of the flow paths may be an inspiratory flow path, configured to deliver the breathable gases flow to the patient; and another one of the flow paths may be an expiratory flow path, configured to receive expiratory gases flow from the patient.

A plurality of the gas flow paths may be inspiratory flow paths.

A plurality of the gas flow paths may be expiratory flow paths.

The gases flow through one gas flow path may be different from the gases flow through another gas flow path.

The gases flow through one gas flow path may be at a higher flow velocity than the gases flow through another gas flow path.

The cross sectional area of one gases flow path may be different from the cross sectional area of another gases flow path.

The cross sectional area of an expiratory flow path may be greater than the cross sectional area of an inspiratory flow path.

The nasal cannula interface may comprise concentric gas flow paths through the prong.

A flow path that is nearer a longitudinal axis of the prong may be configured to transport a higher flow velocity of gases than a flow path that is further from the longitudinal axis of the prong.

Both flow paths may be expiratory flow paths.

A flow path that is nearer a longitudinal axis of the prong may be an expiratory flow path, and a flow path that is further from the longitudinal axis of the prong may be an inspiratory flow path.

A flow path that is nearer a longitudinal axis of the prong may be an inspiratory flow path, and a flow path that is further from the longitudinal axis of the prong may be an expiratory flow path.

The nasal cannula interface may comprise adjacent flow paths through the prong.

The flow paths may be defined by a dividing wall extending along the length of the prong. The dividing wall may divide the prong into flow paths of equal transverse cross section. A plurality of dividing walls may be provided.

The hydraulic diameter and therefore the flow resistance of the inspiratory and expiratory flow paths may be different. The hydraulic diameter of the inspiratory flow path may be higher than that of the expiratory flow path.

The flow resistance of the inspiratory flow path may be lower than that of the expiratory flow path.

The dividing wall may divide the flow path into an anterior flow path and a posterior flow path.

The prongs may be elliptical, when viewed along their longitudinal axis.

In accordance with another aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
c. a porous body comprising porous material;
   wherein the porous body is positioned in the flow path.

The porous body may be configured such that a plug flow velocity profile is delivered from an outlet of the prong into the patient's nare(s).

The porous body may be positioned at least partially within the prong, that is, within the flow path through the prong, so as to plug the prong.

The porous body may be positioned fully within the prong such that the porous body does not extend outside of the prong.

The porous body may be positioned at a tip of the prong so as to cap the prong.

The porous body may be positioned entirely externally of the prong.

The porous body may comprise a diffuser.

The porous body may comprise any one or more of:
a. fabric.
b. woven material.
c. material comprising intertwined strands.
d. knitted material.
e. textile.
f. foam.
g. a material comprising a cellular structure.

The foam may comprise an open cell foam, where the gas flow path is created by the open cells.

The open cell foam may be made from any one or more of: expanded polyethylene, polyurethane, silicone, rubber or the like, fabrics, weaves or cellular structures.

The porous material may comprise:
a. substantially uniform pore distribution;
b. random pore distribution;
c. a combination of uniform and random pore distribution.

In accordance with another aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
c. wherein the flow path is configured such that a plug flow velocity profile is delivered from an outlet of the prong into the patient's nare(s).

The nasal prong may comprise one or more flow guide formations or features in the flow path, to guide the gases flow along the flow path such that plug flow is delivered.

The flow guide formation may comprise a flow vane.

According to a further aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient;
c. wherein the nasal prong comprises an inlet opening configured to receive the gases flow from the inlet of the nasal cannula interface and an outlet opening configured to deliver the gases flow into the patient's nose, the length of the nasal prong between the inlet opening and the outlet opening being sufficient that the nasal prong extends into the nasal cavity of the nose of the patient such that the outlet opening of the nasal prong is beyond the nasal valve of the nose of the patient and/or the nasal prong has a length sufficient to locate the outlet opening in the nasal cavity at or behind the internal nasal valve of the patient.

The flow path through the nasal prong may be between 4 and 30 mm long.

According to another aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient;
c. wherein the nasal prong defines a flow path through the nasal prong between an inlet opening of the prong and an outlet opening of the prong, the cross sectional area of the flow path being greater at or adjacent the prong outlet opening than at or adjacent the prong inlet opening.

The prong may be outwardly flared at the outlet opening.

The cross sectional area of the flow path may increase along the length of the prong.

The cross sectional area may increase over at least half the length of the prong.

The length of the nasal prong may be such that the nasal prong extends sufficiently into the nasal cavity of the nose of the patient such that the outlet of the nasal prong is beyond the nasal valve of the nose of the patient.

The nasal prong may be arcuate.

The nasal cannula interface may comprise a manifold, the nasal prong extending from the manifold, the manifold comprising a lip contacting portion, the nasal prong being arcuate, wherein the lip contacting portion of the manifold and the curve of the arcuate prong, are configured such that the prong engages part of the nares of the patient, whilst leaving a clearance between the remainder of the prong and the nares for an expiratory gases flow.

According to a further aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient at least one gases flow path for the gases flow being provided from the inlet and through the prong;
   wherein the nasal cannula interface further comprises a moisture reducing device configured to be located in the flow path to remove moisture from or reduce moisture in, the patient's nare(s).

The moisture reducing device may comprise one or more hydrophilic, moisture absorbent and/or wicking devices.

The one or more hydrophilic, moisture absorbent and/or wicking devices may be on, at or in the nasal prong.

The moisture reducing device may comprise a hydrophilic, moisture absorbent and/or wicking material.

The nasal cannula interface may comprise a body of said material, the body being mounted on the nasal prong.

The material may comprise an integral part of the nasal cannula interface.

The material may be provided at or on an external surface of the nasal prong.

The material may be provided on one or more other surfaces of the nasal cannula interface.

The device may comprise a moisture flow formation.

The moisture flow formation may comprise a channel, microstructure, capillary or other structure, configured to wick moisture from the patient's nare(s).

The moisture reducing device may comprise a heater.

The heater may be provided on the nasal prong, such as in a wall of the prong, or in the flow path through the prong.

The manifold may comprise the inlet, the nasal prong depending from the manifold, the manifold comprising the heater.

The nasal cannula interface may comprise one or more conduits with which the nasal cannula interface is in fluid communication.

The one or more conduits may comprise the, or a, moisture reducer.

According to another aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
   wherein the nasal prong has an external profile, the nasal prong being adjustable such that a physical characteristic of the nasal prong can be adjusted to alter the external profile of the nasal prong.

The nasal prong may comprise a malleable material.

The nasal prong may comprise an adjustable element, adjustment of the adjustable element adjusting the external profile of the nasal prong.

The adjustable element may comprise a malleable element.

The adjustable element may comprise one or more scaffolding elements, the scaffolding element(s) supporting the nasal prong, and having an adjustable configuration.

The one or more scaffolding elements may comprise one or more elongate elements.

The one or more elongate elements may be metal wires. At least one of the one or more wires comprises a heating wire.

The one or more elongate elements may be plastic wires.

The nasal prong may be adjustable along all of the length of the nasal prong.

The nasal prong may be adjustable along only a portion of the length of the nasal prong.

The adjustable structure may comprise a concertina or bellows structure.

The nasal cannula interface may be configured to be adjustable in a direction along a central axis of the prong, such that the length of the prong can be adjusted.

The nasal cannula interface may be configured to be adjustable in a direction away from a central axis of the prong such that an angle of the prong relative to the remainder of the nasal cannula interface can be adjusted.

A curvature of the nasal prong may be adjusted.

The curvature of the nasal prong can be adjusted such that the prong comprises a complex curve.

The external profile of the prong may be adjustable between a first stable configuration, and second stable configuration.

The nasal cannula interface may comprise one or more centralising features configured to centralise the nasal prong in a patient's nare.

The centralising feature may comprise a projection on the exterior of the prong, the projection configured to engage the patient's nare.

The projection may comprise one or more of: a rib, or ridge, or dimple.

According to a further aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
   wherein the nasal prong comprises one or more flow altering surface features configured to alter the flow of gases along the nasal prong.

The flow altering surface feature may be on an external surface of the prong.

The flow altering surface feature may be on an internal surface of the prong.

The nasal cannula interface may comprise a plurality of flow altering surface features.

The nasal cannula interface may comprise one or more elongate grooves.

The one or more elongate grooves may extend helically around at least part of the prong.

The flow altering surface feature may be configured to vary friction between the prong and the gases flow.

The flow altering surface may comprise one or more dimples.

The flow altering surface may comprise one or more projections.

The one or more projections may comprise a rib or ridge or dimple.

The prong may comprise at least a portion of increased surface roughness.

The portion of increased surface roughness may comprise a coating.

The exterior of the prong may be textured or comprises a textured portion.

The interior of the prong may be textured or comprises a textured portion.

The nasal cannula interface may comprise a tube inside the prong to divide the gases flow path through the prong into a plurality of flow paths, the tube being configured such the gases flow path through the tube conveys a portion of the gases flow at a higher velocity than the portion of the gases flow outside of the tube.

According to another aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
   wherein the flow path is configured such that the gases flow through the flow path comprises a higher velocity component, adjacent a longitudinal axis of the nasal prong, and a lower velocity component radially spaced from the longitudinal axis.

The nasal cannula interface may comprise a tube inside the prong to divide the gases flow path through the prong into a plurality of flow paths, the tube being configured such the gases flow path through the tube conveys a portion of the gases flow at a higher velocity than the portion of the gases flow outside of the tube.

According to a further aspect of this disclosure there is provided a nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient;
   wherein the nasal prong defines a plurality of separate gas flow paths extending through the prong, one of the flow paths comprising an inspiratory flow path for delivering the gases flow to the patient's nare through an inspiratory outlet of the prong; another flow path comprising an expiratory flow path for receiving expiratory gases from the patient through an expiratory inlet of the prong; wherein the expiratory outlet extends further into the patient's nare than the inspiratory outlet.

The expiratory flow path may be longer than the inspiratory flow path.

The flow paths may be concentric.

The flow paths may be adjacent.

The nasal cannula may comprise a plurality of nasal prongs.

The nasal cannula interface may comprise a pair of nasal prongs.

The prongs, or at least one prong, may be a non-sealing prong, configured not to seal with the nare of the patient.

The prongs, or at least one prong, may be a sealing prong, configured to seal with the nare of the patient.

The prongs, or at least one prong, may be a nasal pillow.

The nasal cannula interface may comprise a manifold, the inlet being provided in the manifold, the or each nasal prong depending from the manifold.

The manifold may be elongate, and comprises a longitudinal axis, the longitudinal axis of the manifold being substantially perpendicular to the longitudinal axis of the, or at least one prong.

The manifold may comprise opposed ends, at each side of the manifold when the nasal cannula interface is viewed perpendicularly to the longitudinal axis, at least one end comprising the inlet.

Each end may comprise an inlet.

The nasal cannula interface may comprise a plug configured to selectively close one or other inlet.

The nasal cannula interface may comprise an inspiratory gases inlet conduit, the conduit being configured to sealingly connect with the inlet to deliver the gases flow from the conduit to the inlet.

The nasal cannula interface may comprise a frame, the manifold being mounted on the frame.

The manifold may be removably mounted on the frame.

The frame may comprise a pair of laterally outwardly extending frame arms, the frame arms being provided with headgear connectors.

The frame arms may be twisted along their length.

The or at least one prong may be substantially straight.

The or at least one prong may be arcuate, or at least comprises an arcuate portion.

The nasal cannula interface may comprise a pair of prongs, the prongs being inclined towards one another, when the nasal cannula interface is viewed from the front.

The cross sectional area, shape and/or size of the flow path through the or at least one prong may vary along the length of the prong.

The or each nasal prong may comprise a prong inlet configured to receive the breathable gases flow, and a prong outlet configured to deliver the breathable gases flow to the patient's nare(s), the prong outlet being larger than the prong inlet.

The or each nasal prong may comprise an inlet configured to receive the breathable gases flow, and an outlet configured to deliver the breathable gases flow to the patient's nare(s), the prong inlet being a different shape from the prong outlet.

At least a portion of the flow path through the or at least one prong may be circular.

At least a portion of the flow path through the or at least one prong may be elliptical.

The nasal prongs may be different lengths.

The nasal cannula interface may comprise a plurality of flow paths through the prong, wherein the flow paths are of different lengths.

The or at least one prong may comprise a filter and/or a diffuser.

The nasal cannula interface may comprise:
a. a nasal cannula interface;
b. a nasal pillows interface;
c. a combination interface, comprising a nasal cannula and a cushion configured to seal against the patient's face.

According to an aspect of this disclosure, there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient; and
at least one expiratory gases flow feature configured to allow expiratory gases from the patient to flow through or around the nasal cannula interface, and to control and/or adjust the direction and/or velocity of the expiratory gases flow.

The expiratory gases flow feature may comprise at least one expiratory gases flow channel configured to allow expiratory gases from the patient to flow through the flow channel so as to control and/or adjust the direction and/or velocity of the expiratory gases flow.

The expiratory gases flow feature may comprise a plurality of expiratory gases flow channels.

The expiratory gases flow channel may be provided centrally on the nasal cannula interface. The expiratory gases flow channel may be provided laterally on the nasal cannula interface, spaced away from the centre of the nasal cannula interface. The expiratory gases flow channel may be provided adjacent the nasal prong.

The expiratory gases flow feature may comprise at least one expiratory gases flow protrusion configured such that expiratory gases from the patient flows into, over and/or around the flow protrusion so as to control and/or adjust the direction and/or velocity of the expiratory gases flow. A plurality of expiratory gases flow protrusions may be provided.

The nasal cannula interface may comprise a manifold, the inlet being provided in the manifold, the or each nasal prong depending from the manifold; wherein the manifold does not extend laterally further than the lateral most portion of the prongs.

The manifold may comprise an elongate body having a longitudinal axis, the longitudinal axis being positioned substantially vertically.

The expiratory gases flow feature may comprise a textured surface portion positioned adjacent the prongs such that expiratory gases flow through and/or around the nasal cannula interface flows onto the textured surface.

The textured surface portion may comprise:
a. one or more recesses.
b. one or more protrusions.

The recesses and/or protrusions may be elongate. The recesses and/or protrusions may be arcuate.

The or each nasal prong may comprise an outlet; the nasal cannula interface comprising an additional outlet through which a portion of the breathable gases flow exits the nasal cannula interface; wherein the additional outlet is positioned on the nasal cannula interface to adjust the direction and/or velocity of an expiratory gases flow through or around the nasal cannula interface.

A plurality of additional outlets may be provided.

The or each additional outlet may be elongate. The or each additional outlet may be arcuate.

According to an aspect of this disclosure, there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient; and
at least one expiratory gases flow channel configured to allow expiratory gases from the patient to flow through the flow channel so as to control and/or adjust the direction and/or velocity of the expiratory gases flow.

According to an aspect of this disclosure, there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient; and
at least one expiratory gases flow protrusion configured such that expiratory gases from the patient flows into, over and/or around the flow protrusion so as to control and/or adjust the direction and/or velocity of the expiratory gases flow.

According to an aspect of this disclosure, there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient;
a manifold, the inlet being provided in the manifold, the or each nasal prong depending from the manifold;
wherein the manifold does not extend laterally further than the lateral most portion of the prongs.

According to an aspect of this disclosure, there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient; wherein the nasal cannula interface comprises a textured surface portion positioned adjacent the prongs such that expiratory gases flow through and/or around the nasal cannula interface flows onto the textured surface.

According to an aspect of this disclosure, there is provided a nasal cannula interface for a respiratory support system configured to receive a breathable gases flow, the nasal cannula interface comprising:
an inlet to receive the gases flow;
at least one nasal prong configured to receive the gases flow from the inlet, and to be received in, and to deliver the gases flow to, a nare of the patient via an outlet of the nasal prong; and
an additional outlet through which a portion of the breathable gases flow exits the nasal cannula interface; wherein the additional outlet is positioned on the nasal cannula interface to adjust the direction and/or velocity of an expiratory gases flow through or around the nasal cannula interface.

According to an aspect of this disclosure there is provided a respiratory support apparatus comprising the nasal cannula interface of any one of the above statements, the apparatus comprising a flow generator configured to generate the gases flow.

The apparatus may be a high flow apparatus configured to generate a high gases flow.

The apparatus may comprise any one or more of:
a. a humidifier configured to humidify the gases flow;
b. an inspiratory conduit configured to be connected to the nasal cannula interface and to receive the gases flow from the flow generator or humidifier;
c. an expiratory conduit configured to be connected to the nasal cannula interface to receive expiratory gases from the nasal cannula interface.
d. a controller configured to control the flow generator and/or the humidifier;
e. a user interface configured to enable the patient to control one or more aspects of the system.

The inspiratory conduit may comprise one or more heating elements.

According to another aspect of this disclosure there is provided a nasal cannula interface assembly comprising:
a. the nasal cannula interface of any one of the above statements;
b. headgear configured to be secured to the nasal cannula interface and to secure the nasal cannula interface to the patient's head;
c. an inlet conduit configured to be fluidly connected to the nasal cannula interface.

Features from one or more embodiments or configurations may be combined with features of one or more other embodiments or configurations. Additionally, more than one embodiment may be used together during a process of respiratory support of a patient.

The term ‘comprising’ as used in this specification means ‘consisting at least in part of’. When interpreting each statement in this specification that includes the term ‘comprising’, features other than that or those prefaced by the term may also be present. Related terms such as ‘comprise’ and ‘comprises’ are to be interpreted in the same manner

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

This disclosure may also be said broadly to consist in the parts, elements and features referred to or indicated in the specification of the application, individually or collectively, and any or all combinations of any two or more said parts, elements or features.

To those skilled in the art to which the disclosure relates, many changes in construction and widely differing embodiments and applications of the disclosure will suggest themselves without departing from the scope of the disclosure as defined in the appended claims. The disclosures and the descriptions herein are purely illustrative and are not intended to be in any sense limiting. Where specific integers are mentioned herein which have known equivalents in the art to which this disclosure relates, such known equivalents are deemed to be incorporated herein as if individually set forth.

### BRIEF DESCRIPTION OF THE DRAWINGS

FIG. 1A shows in diagrammatic form a respiratory support apparatus being a High Flow apparatus.

FIG. 1B is a schematic view of another respiratory support apparatus including a nasal cannula interface being a nasal cannula interface.

FIG. 2 is a perspective view of a nasal cannula interface assembly being a nasal cannula assembly including headgear and a breathable gases inlet conduit.

FIG. 3 is a schematic sectional view showing anatomical features of the human nose.

FIGS. 4a) and b) are perspective views showing a nasal cannula interface in accordance with this disclosure, at least partially shaped to better match the contours of a patient's nares, FIGS. 4c and 4d are sectional side views showing a nasal cannula interface in accordance with this disclosure, with one or more features that enable the shape of the nasal cannula to be adjusted, and FIG. 4e is a sectional side view of a nasal cannula interface in accordance with this disclosure that is shaped to contact a wall of a nare of a patient.

FIG. 5a is a sectional side view of a known nasal cannula interface showing a prong of the cannula in a nare of a patient; FIGS. 5b and 5c are sectional side views of longer prongs of a nasal cannula interface in accordance with this disclosure; FIG. 5d is a sectional side view of a known nasal cannula interface; and FIG. 5e is a sectional side view of prongs of a nasal cannula interface in accordance with this disclosure where the outlet of the prongs is shaped to control the flow of breathable gases from the prongs.

FIG. 6A is a sectional side view of a known nasal cannula interface showing a prong of the cannula in the nare of a patient; FIGS. 6B and 6C are sectional side views of a nasal cannula interface in accordance with this disclosure, comprising a porous material, with each Figure also comprising a respective gases flow profile, the flow profile being shown across the flow path through the prong, and FIG. 6d is a sectional side view of a nasal cannula interface in accordance with this disclosure comprising prongs provided with a plurality of flow directing formations.

FIGS. 7a and 7b are perspective views of a nasal cannula interface in accordance with this disclosure, comprising wicking material, with FIG. 7b also showing that an inlet conduit connected to the nasal cannula interface comprises a heater wire.

FIGS. 8a to 8c are sectional side views of a nasal cannula interface in accordance with this disclosure comprising prongs provided with modified internal surfaces.

FIG. 9 is a sectional side view of a nasal cannula interface in accordance with this disclosure comprising prongs provided with an internal flow tube.

FIGS. 10a and 10b are schematic perspective views of a nasal prong of a nasal cannula interface in accordance with this disclosure, comprising an internal flow tube.

FIGS. 11a and 11b are schematic views along the longitudinal axis of a nasal prong of a nasal cannula interface in accordance with this disclosure.

FIGS. 12a to 12e are sectional side views of a nasal cannula interface in accordance with this disclosure, comprising prongs provided with separate inspiratory and expiratory gases flow paths.

FIG. 13 is a perspective view of another nasal cannula interface in accordance with this disclosure, with a central channel for expiratory gases flow.

FIGS. 14a and 14b are an enlarged top view and a perspective view of another nasal cannula interface in accordance with this disclosure, with central and lateral channels for expiratory gases flow.

FIG. 15 is a perspective view of another nasal cannula interface in accordance with this disclosure, with a relatively narrow cannula body.

FIG. 16 is a perspective view of another nasal cannula interface in accordance with this disclosure, with a formation for redirecting expiratory gases flow.

FIG. 17 is a perspective view of another nasal cannula interface in accordance with this disclosure, with a formation for reducing the velocity of expiratory gases flow.

FIG. 18 is a perspective view of another nasal cannula interface in accordance with this disclosure, with an outlet port for inspiratory gases flow.

FIG. 19 is a perspective view of another nasal cannula interface in accordance with this disclosure, with an outlet port for inspiratory gases flow.

FIG. 20 is a perspective view of another nasal cannula interface in accordance with this disclosure, with a plurality of outlet ports for inspiratory gases flow.

### DETAILED DESCRIPTION

It is known in the art to provide a CPAP/Bi-Level respiratory support apparatus comprising a flow generator in the form of a blower which draws ambient air into an impeller to generate an inspiratory gases flow which is delivered to the patient via one or more inspiratory conduits and a nasal cannula interface mounted on the head of the patient. Such apparatus often comprises a humidifier, downstream of the blower, to humidify the inspiratory gases flow prior to delivery to the patient. In some examples, the inspiratory conduit can be heated. Such a respiratory support apparatus can be controlled for example to a user (e.g. clinician) set pressure of 10 cmH₂O, generating an example inspiratory gases flow at a flow rate of around 40 L/min. When controlled to a pressure of 20 cmH₂O, an example inspiratory gases flow might be generated at a flow rate of around 55 L/min.

It is also known to provide a High Flow respiratory support apparatus. One example of such a high flow respiratory support apparatus comprises a nasal cannula interface being a nasal cannula interface comprising prongs received in the nares of the patient. In such an apparatus the patient typically exhales directly to atmosphere via their mouth, and/or via leak around the prongs of the cannula. In such an example such a respiratory support apparatus can be controlled to a user defined flow rate, generating an example inspiratory gases flow at a flow rate of around 50-60 L/min

In the above examples, the pressures and flow rates are examples only, to illustrate the type of respiratory support (i.e. respiratory therapy) that such apparatus can deliver to a patient. Further, more detailed examples are provided below.

### Respiratory Support Apparatus

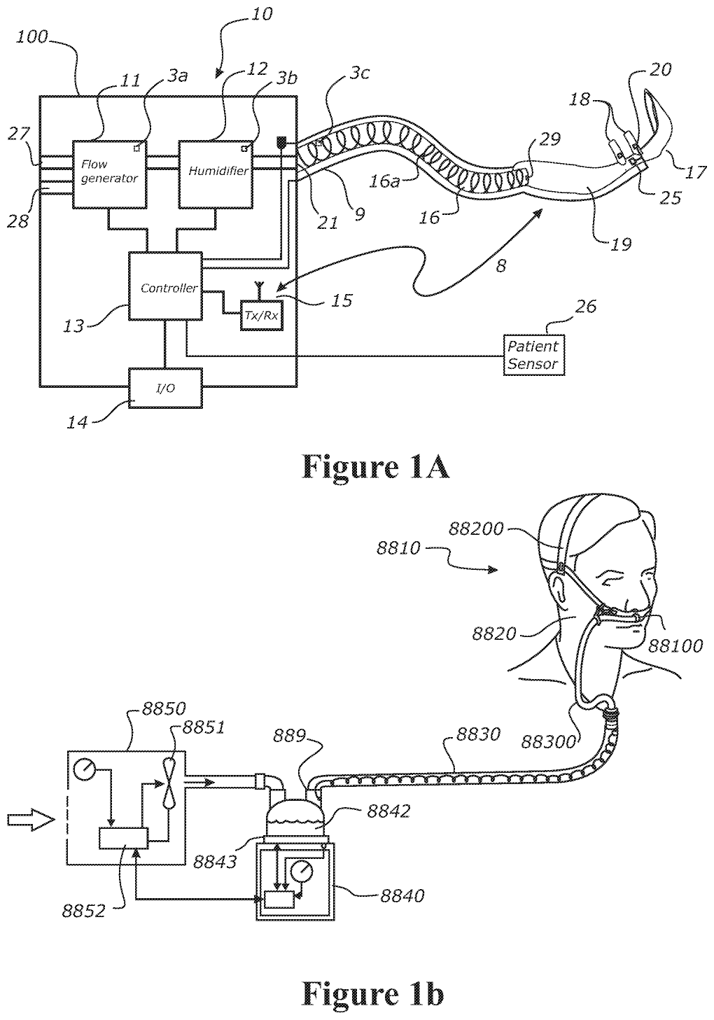

A respiratory support apparatus 10 is shown in FIG. 1A. The respiratory support apparatus 10 can comprise a main housing 100 that contains a flow generator 11 in the form of a motor/impeller arrangement (for example, a blower), an optional humidifier 12, a controller 13, and a user interface 14 (comprising, for example, a display and input device(s) such as button(s), a touch screen, or the like). The controller 13 can be configured or programmed to control the operation of the apparatus. For example, the controller can control components of the apparatus, including but not limited to: operating the flow generator 11 to create a flow of gas (gases flow) for delivery to a patient, operating the humidifier 12 (if present) to humidify and/or heat the generated gases flow, control a flow of oxygen into the flow generator blower, receiving user input from the user interface 14 for reconfiguration and/or user-defined operation of the apparatus 10, and outputting information (for example on the display) to the user. The user can be a patient, healthcare professional, or anyone else interested in using the apparatus. As used herein, a “gases flow” can refer to any flow of gases that may be used in the breathing assistance or respiratory device, such as a flow of ambient air, a flow comprising substantially 100% oxygen, a flow comprising some combination of ambient air and oxygen, and/or the like.

A patient breathing or inspiratory conduit 16 is coupled at one end to a gases flow outlet 21 in the housing 100 of the respiratory support apparatus 10. The patient breathing conduit 16 is coupled at another end to a nasal cannula interface 17 such as a non-sealing nasal cannula interface with a manifold 19 and nasal prongs 18 depending from the manifold 19. The nasal cannula interface 17 is a non-sealing interface in order to provide high flow respiratory support to a patient. The gases flow that is generated by the respiratory support apparatus 10 may be humidified and delivered to the patient via the patient conduit 16 through the cannula interface 17. The patient conduit 16 can have a heater wire 16a to heat gases flow passing through to the patient. The heater wire 16a can be under the control of the controller 13. The patient conduit 16 and/or nasal cannula interface 17 can be considered part of the respiratory support apparatus 10, or alternatively peripheral to it. The respiratory support apparatus 10, breathing conduit 16, and nasal cannula interface 17 together can form a respiratory support system.

The controller 13 can control the flow generator 11 to generate a gases flow of the desired flow rate and/or pressure. The controller 13 can also control a supplemental oxygen inlet to allow for delivery of supplemental oxygen, the humidifier 12 (if present) can humidify the gases flow and/or heat the gases flow to an appropriate level, and/or the like. The gases flow is directed out through the patient conduit 16 and cannula 17 to the patient. The controller 13 can also control a heating element in the humidifier 12 and/or the heating element 16a in the patient conduit 16 to heat the gas to a desired temperature for a desired level of therapy and/or level of comfort for the patient. The controller 13 can be programmed with or can determine a suitable target temperature of the gases flow.

Operation sensors 3a, 3b, 3c, such as flow, temperature, humidity, and/or pressure sensors can be placed in various locations in the respiratory support apparatus 10. Additional sensors (for example, sensors 20, 25) may be placed in various locations on the patient conduit 16 and/or cannula 17 (for example, there may be a temperature sensor 29 at or near the end of the inspiratory tube). Output from the sensors can be received by the controller 13, to assist the controller in operating the respiratory support apparatus 10 in a manner that provides suitable therapy. In some configurations, providing suitable therapy includes meeting a patient's peak inspiratory demand. The apparatus 10 may have a transmitter and/or receiver 15 to enable the controller 13 to receive signals 8 from the sensors and/or to control the various components of the respiratory support apparatus 10, including but not limited to the flow generator 11, humidifier 12, and heater wire 16a, or accessories or peripherals associated with the respiratory support apparatus 10. Additionally, or alternatively, the transmitter and/or receiver 15 may deliver data to a remote server or enable remote control of the apparatus 10.

The respiratory support apparatus 10 may comprise a high flow respiratory support apparatus. High flow respiratory support as discussed herein is intended to be given its typical ordinary meaning as understood by a person of skill in the art, which generally refers to a respiratory assistance system delivering a targeted flow of humidified respiratory gases via an intentionally non-sealing nasal cannula interface with flow rates generally intended to meet or exceed inspiratory flow of a patient. Typical nasal cannula interfaces include, but are not limited to, a nasal or tracheal nasal cannula interface. Typical flow rates for adults often range from, but are not limited to, about fifteen liters per minute (LPM) to about seventy liters per minute or greater. Typical flow rates for pediatric patients (such as neonates, infants and children) often range from, but are not limited to, about one liter per minute per kilogram of patient weight to about three liters per minute per kilogram of patient weight or greater. High flow respiratory support (i.e. high flow therapy) can also optionally include gas mixture compositions including supplemental oxygen and/or administration of therapeutic medicaments. High flow respiratory support is often referred to as nasal high flow (NHF), humidified high flow nasal cannula (HHFNC), high flow nasal oxygen (HFNO), high flow therapy (HFT), or tracheal high flow (THF), among other common names. The flow rates used to achieve “high flow” (i.e. high flow respiratory support) may be any of the flow rates listed below. For example, in some configurations, for an adult patient ‘high flow respiratory support’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between 25 LPM and 75 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow respiratory support’ (i.e. high flow therapy) may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow respiratory support apparatus with an adult patient, a neonatal, infant, or child patient, may deliver gases to the patient at a flow rate of between about 1 LPM and about 100 LPM, or at a flow rate in any of the sub-ranges outlined above. The respiratory support apparatus 10 can deliver any concentration of oxygen (e.g., FdO2), up to 100%, at any flowrate between about 1 LPM and about 100 LPM. In some configurations, any of the flowrates can be in combination with oxygen concentrations (FdO2s) of about 20%-30%, 21%-30%, 21%-40%, 30%-40%, 40%-50%, 50%-60%, 60%-70%, 70%-80%, 80%-90%, and 90%-100%. In some combinations, the flow rate can be between about 25 LPM and 75 LPM in combination with an oxygen concentration (FdO2) of about 20%-30%, 21%-30%, 21%-40%, 30%-40%, 40%-50%, 50%-60%, 60%-70%, 70%-80%, 80%-90%, and 90%-100%. In some configurations, the respiratory support apparatus 10 may include safety thresholds when operating in manual mode that prevent a user from delivering to much oxygen to the patient.

High flow respiratory support may be administered to the nares of a user. High flow respiratory support may deliver gases to a user at a flow rate at or exceeding the intended user's peak inspiratory flow requirements. The high flow respiratory support may generate a flushing effect in the nasopharynx such that the anatomical dead space of the upper airways is flushed by the high incoming gases flow. This can create a reservoir of fresh gas available for each and every breath, while minimizing re-breathing of nitrogen and carbon dioxide. Meeting inspiratory demand and flushing the airways is additionally important when trying to control the patient's FdO2. High flow respiratory support can be delivered with a non-sealing nasal cannula interface such as, for example, a nasal cannula. The nasal cannula may be configured to deliver breathing gases to the nares of a user at a flow rate exceeding the intended user's peak inspiratory flow requirements.

The term “non-sealing nasal cannula interface” as used herein can refer to an interface providing a pneumatic link between an airway of a patient and a gases flow source (such as from flow generator 11) that does not completely occlude the airway of the patient. Non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or more of a nare or mouth of the patient. For a nasal cannula interface the airway is through the nares.

The flow generator or blower 11 can include an ambient air inlet port 27 to entrain ambient room air into the blower. The respiratory support apparatus 10 may also include an oxygen inlet port 28 leading to a valve through which a pressurized gas may enter the flow generator or blower 11. The valve can control a flow of oxygen into the flow generator blower 11. The valve can be any type of valve, including a proportional valve or a binary valve.

The blower can operate at a motor speed of greater than about 1,000 RPM and less than about 30,000 RPM, greater than about 2,000 RPM and less than about 21,000 RPM, greater than about 4,000 RPM and less than about 19,000 RPM or between any of the foregoing values. Operation of the blower can mix the gases entering the blower through the inlet ports. Using the blower as the mixer can decrease the pressure drop that would otherwise occur in a system with a separate mixer, such as a static mixer comprising baffles, because mixing requires energy. Having a static mixer can also increase the volume of the gas flow path between the valve and the gases composition sensor, which can further increase the delay between when the valve current is changed and when a corresponding change in oxygen concentration is measured.

Based on user inputs and the therapy supplied by the specific device, the controller can determine a target output parameter for the blower. The controller can receive measurements of the target output parameter, and based on the difference between determined flow rate and the measured flow rate, the controller can adjust the speed of the blower.

The target output parameter may be flow rate. The target flow rate may be a constant value, (e.g., nasal high flow). The target flow rate may be a value that fluctuates. In some configurations, the controller can control a blower motor speed based on a target flow rate, and additionally increase or decrease the motor speed based on a patient's breathing cycle. The target flow rate does not necessarily change, but, the controller causes the motor speed to fluctuate in order add oscillations to the instantaneous flow rate such that the flow rate is synchronized with the patient's breathing. Such a system is described in International Application No. PCT/NZ2017/050063, titled “Flow Path Sensing for Flow Therapy Apparatus”, filed on May 17, 2017.

The target output parameter may alternatively be pressure. The target pressure may be a constant value, (e.g., CPAP). Alternatively, the target flow rate may a value that fluctuates, potentially in time with the breath, (e.g., bi-level NIV). In both of these scenarios, the total flow rate is unlikely to be constant.

Referring to FIG. 1B, a respiratory support apparatus 8810 as may be used with the present disclosure is shown. In such a apparatus 8810, a patient 8820 is supplied with a humidified flow of gases through a nasal cannula interface 88100. The nasal cannula interface 88100 is retained in an operational position upon the patient's face using associated headgear 88200. The interface 88200 is connected to a humidified gases transportation pathway or inspiratory conduit 8830. The inspiratory conduit 8830 is connected 5 at one end (either directly or indirectly) to the nasal cannula interface 88100 and at an opposing end to the outlet of a humidifier 8840. In the preferred embodiment the inspiratory conduit is connected to the nasal cannula interface via an extension tube/conduit 88300. The humidifier 8840 receives and humidifies gas supplied from a gases supply source 8850, preferably including a blower 8851. The humidifier 8840 may comprise a humidification chamber 8841 filled with water 8842 and a heating means 8843 for heating the water to humidify the gas path through the humidifier.

A controller 8852 may be provided to control and possibly vary one or more properties of the supplied gas, including but not limited to the pressure profile of the gas, the flow rate profiles of the gas at the nasal cannula interface, the temperature of the gas and/or the humidity of the gas. It will be appreciated that the control capabilities are dependent on the purpose and application of the respiratory system 8810. For example, in an application of in-hospital respiratory care, the flow rate of supplied gas is monitored and controlled according to the patient's requirements, but the pressure of the supplied gas is not necessarily monitored and controlled. In alternative embodiments, such as the use of the disclosure in CPAP, the pressure profile of the supplied gas may be monitored and controlled.

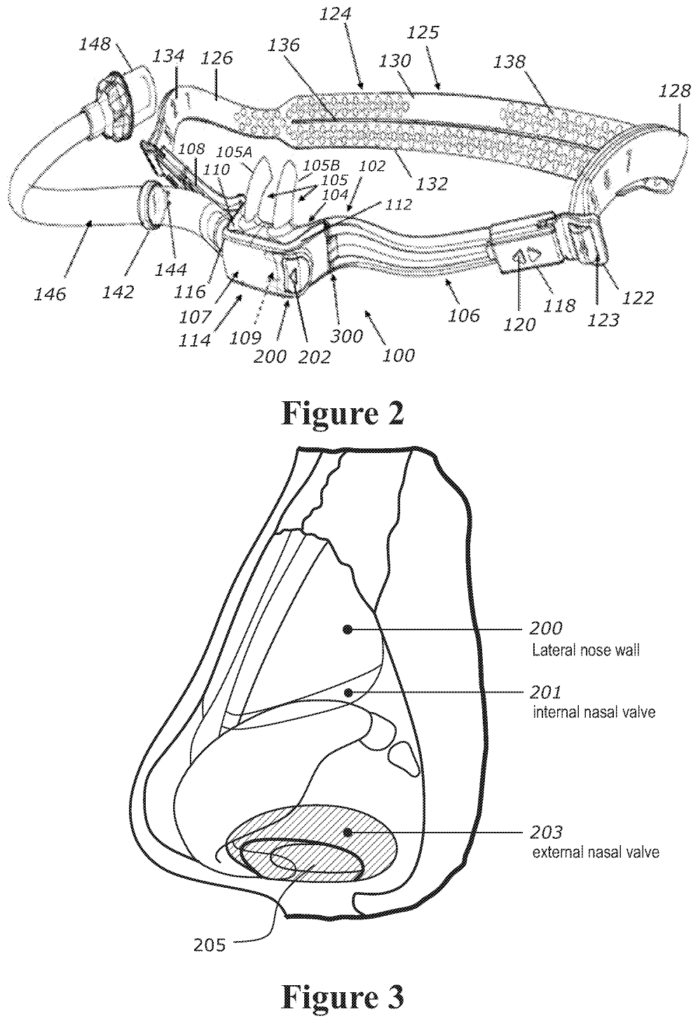

With reference to FIG. 2, a nasal cannula interface can comprise a nasal cannula interface, as described in our earlier patent application WO2015/193833 published 23 Dec. 2015, the entire contents of which are hereby incorporated by reference.

The nasal cannula interface 100 comprises a frame portion 102. The frame portion 102 comprises a contact region 104 that contacts a user in use. At least a part of the contact region 104 sits under a nose or under nares of a user in use (for example, on the lip superior). The frame portion 102 also comprises a non-contact region 107 that faces away from the user in use.

In the illustrated configuration, the non-contact region 107 can be formed from a relatively hard or rigid material (for example polycarbonates and/or polypropylene) that provides support to the frame portion 102. The contact region 104 can comprise a relatively soft, flexible or pliable material (including but not limited to foams, gelbased materials, silicones and/or thermoplastic elastomers) that can be positioned against the face of a user. The contact region 104 can be overmoulded or co-moulded onto the non-contact region 107. Soft or flexible materials can be used in the contact region 104 to increase comfort to the user and may form a soft pillow-like structure that rests in contact with the patient. In some configurations, the contact region 104 may be secured to the non-contact region 107 by other means, including but not limited to adhesives, ultrasonic welding, and/or mechanical fasteners. In some configurations, the frame portion 102 may only comprise a single material.

The frame portion 102 may also comprise a gases chamber 109 within the frame portion 102 that can receive gas from a gas source. Gas received into the gases chamber 109 can be channelled to the user through a gas delivery element 105. The gas delivery element 105 delivers gases to the user substantially or completely through the nose, and comprises first and second nasal delivery elements 105A, 105B, such as nasal prongs that are adapted to be fitted into the nares of the user. In the illustrated configuration, the nasal delivery elements 105A, 105B engage with the nares to form an non-sealing engagement. In some alternative configurations, the nasal delivery elements 105A, 105B may seal 5 with the nares, or the nasal delivery elements 105A, 105B may have different sealing qualities (for example, the first nasal delivery element 105A may be adapted to sealingly engage with one nare and the second nasal delivery element 105B may not sealingly engage with another nare). In further alternative configurations, the gas delivery element 105 may only comprise a single nasal delivery element, or may only comprise one or more apertures that communicate gases to a nasal, oral, and/or tracheal airway of a user. The frame portion 102 and gases chamber 109 thus form a manifold 114, from which the prongs 105A, 105B depend, and which can be connected to an inspiratory conduit.

The frame portion 102 may comprises a first side arm 106 and a second side arm 108. The first and second side arms 106, 108 extend laterally from the frame portion 102 and can help to support the frame portion 102 and generally rest on a patient's face (e.g. on the cheeks or adjacent the cheeks). The side arms 106, 108 comprise headgear retaining mechanisms 118 adapted to hold headgear 124.

The nasal cannula interface 100 also comprises a manifold 114 that receives gas from a gas source through a manifold inlet 116. The manifold inlet 116 is, at least in some orientations or configurations, in pneumatic communication with the gases chamber 109 of the frame portion 102. The manifold inlet 116 communicates with the gas source through a gas conduit 146 that interfaces with the gas source via a gas source connector 148. The gas source connector 148 may be adapted to swivel or engage in rotary motion relative to the conduit 146 and/or to the gas source. In some alternative configurations, the gas source 5 connector 148 may interface with a gas humidifier or other respiratory therapy device that may be in pneumatic communication with the gas source.

Some examples of respiratory support apparatuses are disclosed in International Application No. PCT/NZ2016/050193, titled “Flow Path Sensing for Flow Therapy Apparatus”, filed on Dec. 2, 2016, and International Application No. PCT/IB2016/053761, titled “Breathing Assistance Apparatus”, filed on Jun. 24, 2016, which are hereby incorporated by reference in their entireties. Examples of configurations of respiratory support apparatuses that can be used with aspects of the present disclosure are discussed in further detail below.

With reference to FIG. 3, various anatomical features of a human nose are shown including the lateral nose wall 200, the internal nasal valve 201, the external nasal valve 203, and the nares 205.

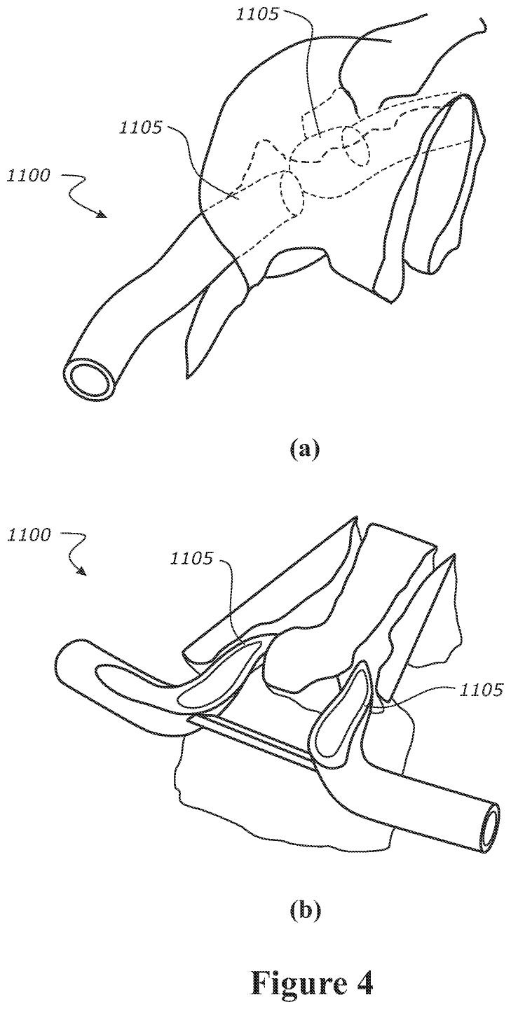

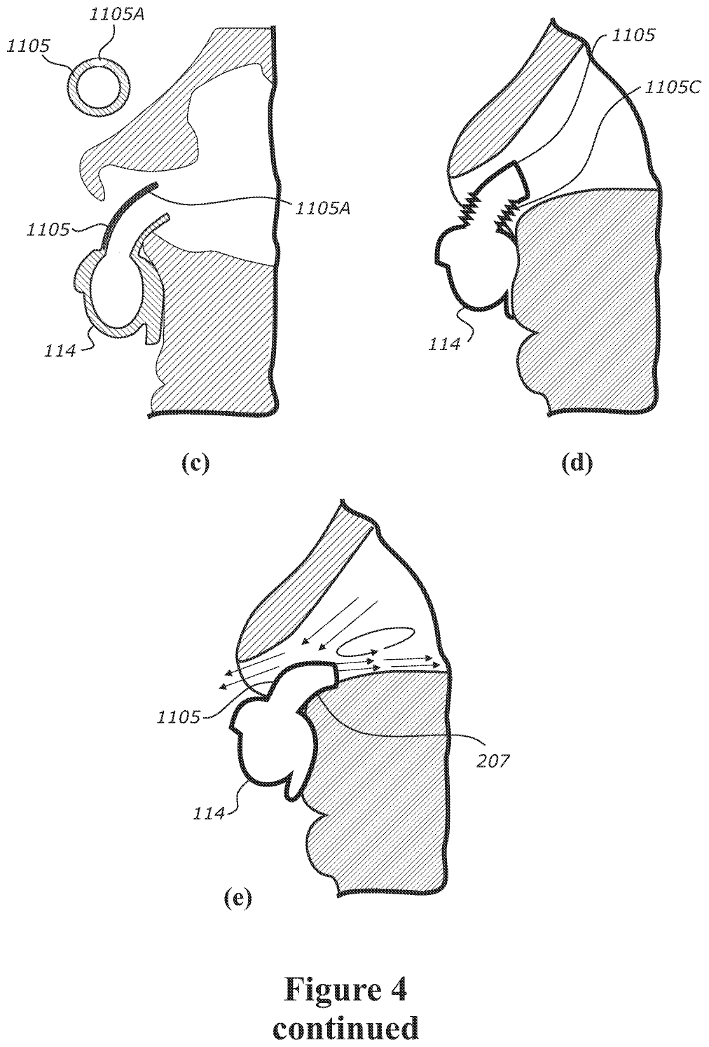

With reference to FIG. 4, an embodiment of a nasal cannula interface 1100 in accordance with this disclosure includes a pair of spaced apart nasal prongs 1105 the external shape of which can be contoured to match the geometry of the nares of the patient. Thus the shape, size and/or surface contours of the prongs 1105 may be altered.

The nasal prongs 1105 may be made of, or comprise one or more additional elements of, malleable material that can be shaped and moulded by the user. In the example of FIGS. 4a and 4b, the prongs 1105 are formed from the malleable material, such that an additional malleable element is not required.

The prongs 1105 may comprise one or more reinforcing elements configured to support the prongs 1105 in an adjusted configuration. The reinforcing elements may be provided internal or externally of the prongs, or within the walls of the prongs.

Malleable material (for example one or more wire/s or other scaffold material or element(s)) could be added to the nasal prongs 1105 to facilitate shaping or contouring to the individual patient geometry as desired. The material may also comprise a heating wire which can be heated to reduce excess moisture. A portion of, or all of, the prong 1105 may comprise the malleable material.

Referring to FIG. 4c, the nasal prongs 1105 are provided with a single malleable elongate element 1105A which extends, in this example, substantially along the length of the prongs 1105. Elongate element 1105A may comprise a metal or plastic wire, or a metal or plastic strip. The wire or strip 1105A in this example is embedded into the wall of the prongs 1105, so as not to interfere with the gases flow path through the prongs 1105, nor alter the external profile of the prongs 1105. Each prong 1105 may be provided with a plurality of such malleable elongate elements 1105A. Elements 1105A may each extend all or only part way along the prongs 1105. Elements 1105A may be equispaced around the prongs 1105 (when viewed along the longitudinal axis of the prongs 1105), or alternatively non-evenly spaced.

Additionally or alternatively, the prongs 1105 may comprise a discrete, adjustable region or regions to also allow radial and/or axial adjustment of the prongs 1105 as desired (relative to the longitudinal axis of the prongs 1105). With reference to FIG. 4d), an example configuration of such an adjustable region is a concertina region 1105C. Concertina region 1105C is provided at or near the base of the prongs 1105 adjacent the manifold 114 of the cannula. Each prong 1105 may be provided with one or more concertina regions 1105C.

Contoured prongs, and/or prongs that are malleable and/or comprise one or more concertina regions can be configured to maintain a preferred distance and/or orientation between the nasal prong 1105 and the nasal cavity wall. This configuration can be achieved by adjusting the position of the prong 1105 in the patient's nare, for example by adjusting the orientation and/or shape of the prongs 1105. This can be achieved by curving one or more portions of the prongs 1105, between the manifold 114 and the prong outlet. The prongs 1105 can be configured to comprise one or more substantially straight portions, and one or more arcuate portions.

A preferred orientation may involve avoiding contact with some or all of the nasal wall which may result in less jetting and lower shear stresses resulting in fewer aerosols being generated. An example preferred prong orientation and shape can be seen in FIG. 4b, where it can be seen that the prongs 1105 do not contact the patient's nares.

This preferred orientation may involve avoiding or at least reducing contact with the nasal wall which may result is less jetting and lower shear stresses resulting in fewer aerosols being generated.

Aerosols, being a suspension of relatively tiny particles or droplets in the air, can be generated through various mechanisms, including the interaction of a high velocity air stream with that of a relatively slow-moving flow of liquid. These tiny particles or droplets can contain undesirable matter being substances and/or contaminants that are undesirable for persons to come in contact with. The aerosols may be infective and/or contain pathogens and, if inhaled or contacted with fluids from the eye, may cause adverse health effects. Patients in respiratory distress may be treated with therapies such as NHF. Such patients may be a source of aerosols carrying undesirable matter. It may be desirable to manage expiratory gases during delivery of high flow therapy with a non-sealing nasal cannula interface, such as NHF, where respiratory gas is jetted into the patient airway at velocities of up to about 70 L/min

Transmission of aerosols can occur through respiratory droplets that may be expelled by a patient, including whilst receiving breathable gases via a nasal cannula interface.

In the case of a virus or other infection disease for example, the risk of transmission can depend on the quantity of viable virus that makes its way to a site of infection in the patient, which in turn can depend on the quantity of virus emitted through respiratory droplets and the survival rate of the virus during transmission.

A key feature of aerosol transmission is the evolution of the size of the respiratory droplets, as this can determine whether the droplets comprise a risk of airborne or droplet transmission. Respiratory droplets come in a range of sizes, characterised by diameters with an upper limit of several hundred micrometers. The droplets evaporate as a function of characteristics such as their size, speed, chemical composition and how they are bunched together in the expiratory ‘jet’. These dependencies arise because evaporation is driven by a difference in partial pressure and temperature at the surface of the evaporating substance and its environment. Accordingly, the external influences on the evaporation can include the room temperature, humidity and ventilation patterns.

Larger droplets can hit the ground or some surface before they completely evaporate. These form the droplet route of infection. The smaller droplets evaporate to become so small (for example less than ˜5 microns) that they linger for several minutes in the air because the effect of gravity is opposed by the drag force they experience. These are called droplet nuclei and represent the airborne route of infection.

Furthermore, avoiding contact with the nasal wall may decrease irritation and decrease sneezing and spreading of aerosols.

External prong locating features such as external ribs or dimples could be present on the outer prong surfaces to help position and maintain the prongs substantially centrally in the nares, and avoid or reduce contact of the prongs 1105 with the nasal wall.

In some cases, prongs 1105 can be configured to contact a wall of the patient's nares. Such controlled contact, in one or more discrete regions of the patient's nares, may be beneficial. The prongs 1105 can be shaped, contoured, oriented and/or positioned to fit the prong 1105 against either the front or back nasal wall. In such a configuration, the gases flow is directed onto that wall and may take advantage of the Coanda effect to direct flow, and any particles generated by the delivery jet, posteriorly. FIG. 4e shows an example configuration in which the prong 1105 is bent, and shaped, to contact the rear nasal wall 207 of the patient's nare.

The radius of curvature of the prongs 1105 may be reduced to reduce flow acceleration on the outer wall (i.e. to reduce centripetal acceleration) and create a more even jet. This can result in more even flow delivery of the gases flow, described below, which can reduce the maximum velocity of flow, which can reduce the aerosols produced.

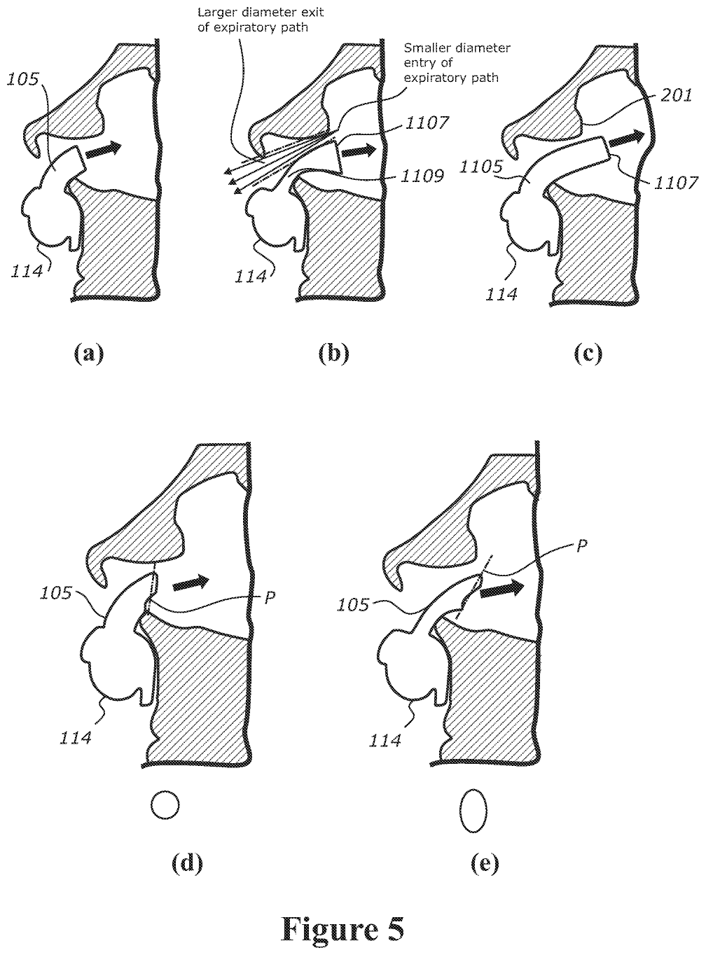

Referring now to FIG. 5, FIG. 5a shows a typical prong 105, for example similar to the prong 105 of FIG. 2, located in a patient's nare. Prong 105 extends into the nare, but terminates prior to the internal nasal valve 201. The prong 105 may have a length, measured along the longitudinal axis of the prong 105, beginning at the manifold 114, and terminating at the prong outlet opening 1107. This length may be equal to or less than the distance from manifold 114 to the patient internal nasal valve 201.

FIG. 5b shows a prong 1105 in accordance with this disclosure in which the prong outlet opening 1107 is larger than the prong inlet opening 1109. The inlet opening 1109 can be defined as the opening, i.e. the transverse cross section of the flow path, where the end of the prong 1105 intersects with the manifold 114. The cross-sectional diameter of the flow path between the prong inlet opening 1109 and prong outlet opening 1107 may smoothly increase towards the prong outlet opening 1105. The outlet end of the prong 1105, and in particular the region of the prong 1105 including the prong outlet opening 1107 is radially outwardly flared, away from the longitudinal axis of the prong 1105.

The diameter (hydraulic diameter) of prongs 1105 can be adjusted. For example, relatively larger prong diameters can lead to a lower inspiratory gases delivery velocity and relatively smaller prong diameters can lead to a greater leakage area and lower expiration gases velocity. Both of these can reduce aerosol generation. A balance between these, and/or an optimal cross-section along the length of the prong 1105, may be achieved to exploit these benefits.

As shown in FIG. 5b, by varying the prong outer diameter along its length, a narrower diameter prong portion 1111 can provide a larger leakage area or expiratory gases flow path. A lower velocity of expiratory gas at the nostril is seen by increasing the prong outer diameter from the manifold 1105 to the prong tip and outlet opening 1107.

The outlet opening 1107 of the prong 1105 may flare out and/or may be sectioned or cut at an angle to further increase the outlet opening diameter, to reduce delivery velocity of the gases flow. This can be seen with reference to FIG. 5d showing a typical prior art prong 105, and FIG. 5e, showing a prong 1105 in accordance with this disclosure. The shaded shapes below each Figure indicate the transverse cross sectional profile of the prong outlet opening 1105 in each case.

With reference to FIG. 5d, the periphery of outlet opening 105C of prior art prong 105 substantially occupies a plane P, the plane P being substantially vertical in normal use. With reference to FIG. 5e, the plane P is substantially inclined in normal use, increasing the size of the outlet opening 1107. The outlet opening 1107 of the prong 1105 is cut at an angle that is more inclined relative to the longitudinal axis A of the prong 1105, has a larger cross sectional area, and reduces the exit velocity of the breathing gases flow from the prong 1105. In other words, the prong 1105 extends further into, is longer, and curves more, than the prong 105.

The outlet opening 1107 of prong 1105 may have a predetermined shape. In one example, the outlet opening 1107 may be tear drop shaped, with the narrow part of the outlet opening 1107 being uppermost.

The cross section of the expiratory path may also increase after the flared region of the prongs 1105 (moving from the flared region towards atmosphere) where there is a reduction in occlusion which results in a lower expiration velocity.

Generally, a larger expiration path i.e. a larger expiration aperture or area results in a reduced expiration velocity, which may reduce the spread of any generated aerosols. This is especially relevant for non-sealing nasal cannula interfaces, where the expiratory path leads to atmosphere.

Due to the nasal cavity shape, it may be beneficial to have a greater change in prong diameter in the vertical direction, away from the manifold 114, to achieve a larger exit diameter (i.e. the prong 1105 flares out in a vertical direction, in normal use of the nasal cannula interface).

Flared prongs 1105 and sectioned prongs 1105 may act as a diffuser, reducing the flow velocity out of the prongs 1105.

FIG. 5c shows a prong 1105 in accordance with this disclosure, which is longer than prong 105, between an inlet opening 1109 and an outlet opening 1107 of the prong 1105. FIG. 5c shows a prong 1105 which extends into the patient's nare and terminates beyond the internal nasal valve 201, that is, prong outlet opening 1107 is beyond the internal nasal valve 201.

The prongs 1005 may be of a length that extends further up the nasal cavity (i.e. past internal nasal valve 201) compared with standard nasal prongs 105.

This results in the inspiratory gases flow exiting the prongs 1105 into a larger cavity in the patient's nose, reducing the turbulence and/or flow interactions in the lower part of the nose that can result in shedding of aerosols from the nasal wall. Generally, aerosols can be generated when the gases flow passes along the internal nasal wall and interacts, disrupts and thus sheds aerosols from the nasal wall into the flow of gases.

The length of an ‘extended prong’ can vary across patient populations e.g. infant/pediatric/adult and various sizes in these groups. In one embodiment, an adult prong may have a gases flow path that is 30 mm long. In one example a neonate prong may have a gases flow path that is 4 mm long.

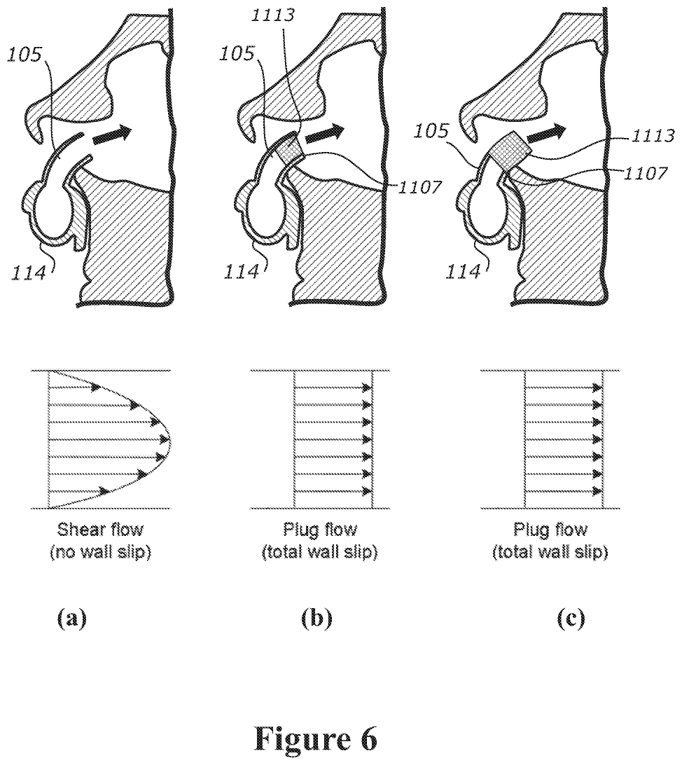

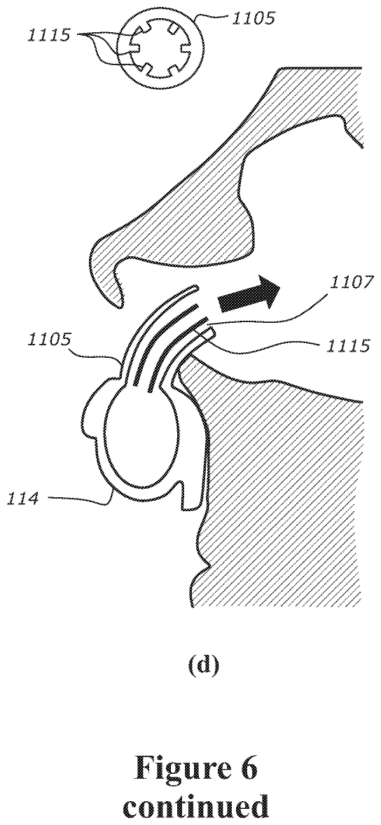

Referring now to FIG. 6, embodiments are described in which the flow profile of the breathable gases flow through the flow path defined by the prong(s) is controlled. FIG. 6a shows a known prong 105, in which the flow path from prong manifold 114 into the prong 105 can be seen, with the flow path through the prong 105 being of substantially constant transverse cross sectional shape and area.

It can be desirable to configure a nasal cannula interface to provide a more even velocity profile as flow exits the nasal prongs 1105 through outlet openings 1107. This can result in less disruption and shedding of aerosols on the nasal walls. In a particularly advantageous configuration of the prongs 1105, the flow profile across the flow path through the prongs would be sufficiently even to provide plug flow.

A porous material present at or in the tip of the nasal prongs 1105, for example in or at least covering the prong outlet opening 1107, can be provided to achieve this even velocity profile. The material may act as a diffuser.

The porous material could comprise a foam, fabric, woven or cellular structure.

The porous material could comprise open cell foam, where the gas flow path is created by the open cells. The open cell foam may be made from expanded polyethylene, polyurethane, silicone, rubber or the like, fabrics, weaves or cellular structures.

The porous material may have substantially uniform pore distribution, random pore distribution, or a combination of both. The pore size can be varied to suit the required purpose.

Referring to FIG. 6b, the porous material may be provided as a body of material 1113 which plugs the distal end of the prong 1105, at the prong outlet opening 1107, that is, the body of material 1113 is fully inserted inside the prong 1105. In this example, the material 1113 does not project beyond the prong outlet opening 1107.

Referring to FIG. 6c the porous material may be provided as a body of material 1113 which caps the distal end of the prong 1105, at the prong outlet opening 1107, that is, the body of material 1113 is external of the prong 1105, but covers the prong outlet opening 1107.

As can be seen from the velocity flow profiles, across the width of the flow path through the prongs, the known prong 105 of FIG. 6a experiences shear flow across the flow path, with the flow adjacent the walls of the prong 105 being lower than the flow adjacent along the longitudinal axis LA of the prong 105, with substantially no wall slip. The prongs 1105 of FIGS. 6b and 6c experience plug flow where the flow is substantially constant across the flow path, with substantially total wall slip.

A portion of the prong 1105 or the entire prong 1105 may comprise the porous material. The length of porous material, that is the length of the body 1113, can vary. A longer length of the same porosity material may reduce velocity. A coarser material, such as foam, of the same length would reduce resistance to flow of the prongs.

The non-porous prong material may or may not cover the circumference of the porous material. The porous material may be able to absorb moisture. The porous material be heated to also wick and evaporate condensate in at or near the prong 1105.

Alternative prong geometries (e.g. flow vanes) may be used to promote even flow. For example, the prong 1105 may be provided with one or more flow guide formations. With reference to FIG. 6d, flow guide formations are provided comprising a plurality of elongate flow vanes 1115 inside the prong 1105, that is, in the flow path through the prong 1105. In this example the flow vanes 1115 are elongate and extend substantially along the length of the prong 1105, parallel to the longitudinal axis A of the prong 1105, each vane 1115 projecting radially inwardly from the prong wall, into the flow path inside the prong 1105. When viewed along the longitudinal axis A of the prong 1105, the flow vanes 1115 may be equispaced around the axis of the prong 1105. Any desired number of flow vanes 1115 may be provided. In the example shown there are six flow vanes 1115 per prong 1105. Other flow guide formations can be provided in the flow path, or which project into the flow path from the wall of the prong 1105. Flow guide formations can be provided which extend along only part of the length of the prongs 1105, or which are provided in only a portion of the prong 1105.

To reduce aerosolisation from excess moisture, a hydrophilic, absorbent or wicking material can be added to the outer surfaces of the nasal prongs to wick away and evaporate condensate and mucus. Alternatively, the nasal prongs may be made of a moisture wicking material. Any surface or all surfaces of the nasal cannula may be made of or comprise the wicking material.

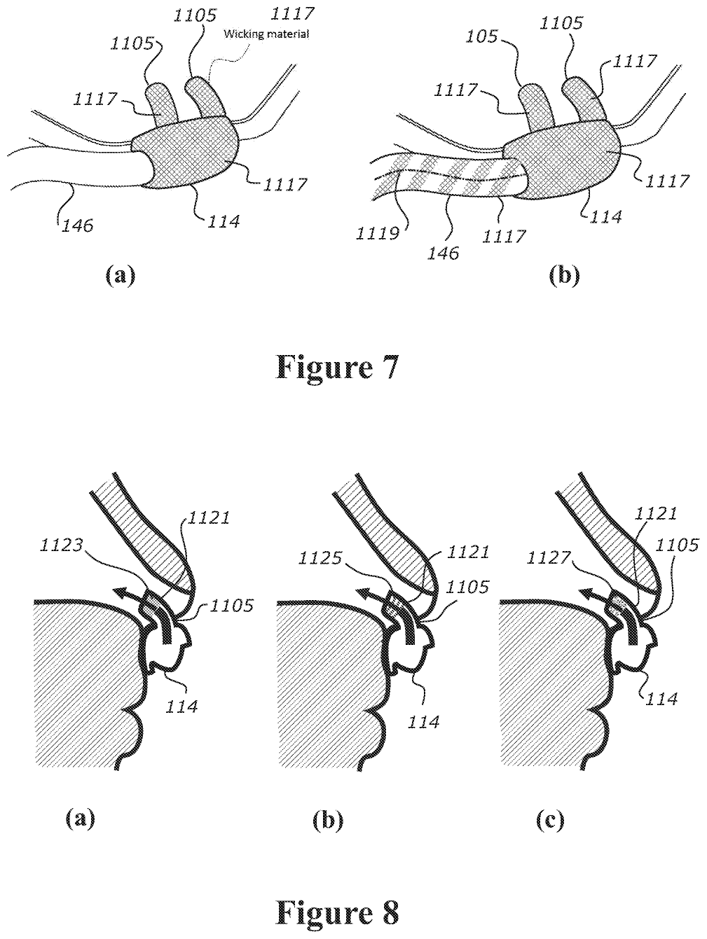

With reference to FIG. 7, a nasal cannula interface is provided with a wicking material to wick moisture from condensate or mucus in the nostril area. This moisture can be a source of aerosolisation. As the expiratory gas flow travels over the moisture, this can disperse moisture droplets into smaller droplets of a size that is relevant for aerosolisation.

In the example of FIG. 7a wicking material 1117 is provided on both prongs 1105, and on the cannula manifold 114. In the example of FIG. 7b, the wicking material 1117 is also provided on the gases inlet conduit 146 that is connected to the manifold 114.

With reference to FIG. 7b a nasal cannula interface is provided comprising wicking material 1117 on both prongs 1105 and the manifold 114, and a heating wire 1119 located in the gases inlet conduit 146, to heat the gases inlet conduit 146 and the inspiratory gases therein. The, or another, heating wire 1119 can be provided in the cannula manifold 114 and/or in one or both prongs 1105. Heating of the inspiratory gases can help reduce condensate from the gases forming in the inspiratory flow path to the patient and/or the expiratory flow path from the patient.

The prongs 1105 may comprise microstructures such as channels/micro-channels or other structures to direct or funnel moisture to wicking materials to evaporate. Such microstructures can take the form as described in our earlier international published patent application WO2014003579, filed 25 Jun. 2013.

The prongs 1105 or nasal cannula manifold 114 may comprise thermally conductive plastic to facilitate heat transfer from, for example, the heated inlet conduit 146, to improve evaporation.

Additional heating along the tubing and/or cannula can decrease condensate formation in the cannula.

Additional heating along the tubing and/or cannula can increase the rate of evaporation from the wicking material.

For example, we provide prongs configured such that a flow profile out of the prongs 1105 comprises a central, high velocity flow region adjacent the longitudinal axis A of the prongs 1105, and low velocity flow surrounding the central, higher velocity jet, and spaced from the longitudinal axis A. Such a configuration can help maintain the benefits of jetting flow whilst minimizing aerosol shedding and generation.

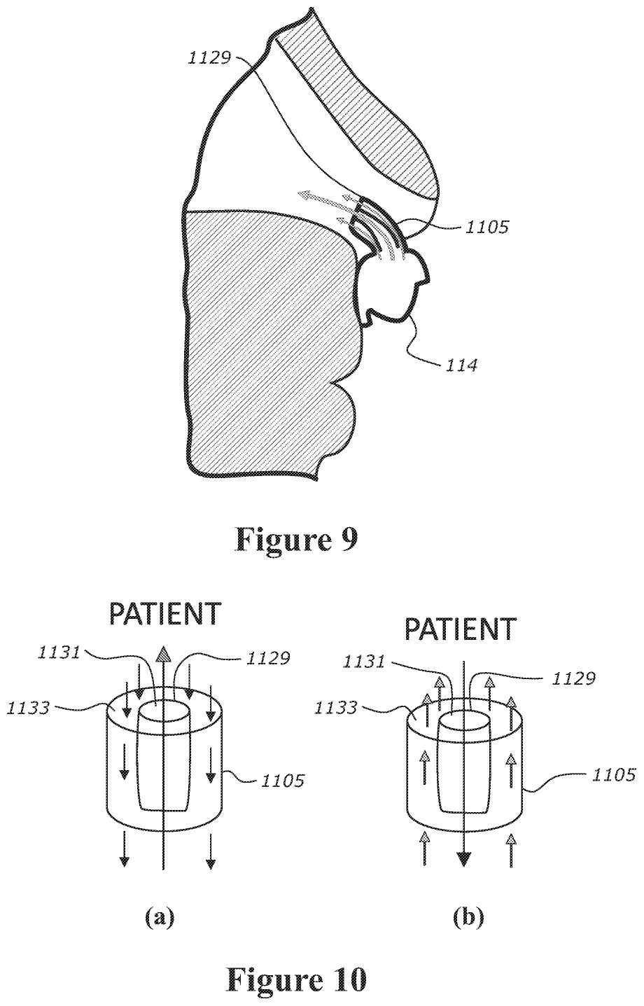

Such a flow profile can be achieved by providing multiple discrete inspiratory flow paths through the prongs 1105 as described below with reference to FIGS. 9 to 12. Such a flow profile can also or additionally be achieved by altering the surface characteristics of some or all of the outer surface of the flow path, that is, by altering the surface characteristics of the internal wall of the prongs 1105, with reference to FIG. 8.

The central high velocity flow provides the benefits of jetting: that is, improved CO2 clearance and gas transfer in the patient. The surrounding low velocity flow may result in a reduction of aerosol shedding.

Providing a higher velocity central flow and a slower velocity outer flow may be achieved by increasing friction on the internal surfaces of the flow path through each prong 1105 to slow the outer flow down. This may be done via a surface coating or surface texturing that varies the surface roughness of these internal surfaces. For example, FIG. 8a shows a prong 1105 where an outlet region 1121 of the prong 1105, distal from the manifold 114, is provided with a coating 1123. In FIG. 8b, the outlet region 1121 of the prong 1105 is provided with texturing in the form of a plurality of circumferential formations 1125, which could be grooves or ribs for example. In FIG. 8c, the outlet region 1121 of the prong 1105 is provided with texturing in the form of a plurality of dimples 1127. The surface texturing could comprise any combination of projections and/or recesses. Examples of such surface texturing include any one or more of, or: dimples, ridges, grooves; whether straight or arcuate or both.

Referring to FIGS. 9 to 12, embodiments are shown wherein the or each prong 1105 comprises a plurality of gases flow paths through the prong 1105. Each prong 1105 can therefore comprise an inspiratory flow path and an expiratory flow path, or multiple inspiratory flow paths, or multiple expiratory flow paths. Providing multiple flow paths can allow the gases flow to be desirably controlled or varied.

With reference to FIG. 9, additionally, or alternatively a central tube 1129 may be present in the or each prong 1105 and the velocity of flow that exits this tube will be higher than surrounding flow. In this example the central tube 1129 is coaxial with the longitudinal axis of the prong 1105. The central tube 1129 converges along the length of the prong 1105 such that the tube outlet 1129A is smaller than the tube inlet 1129B. This convergence of the flow path through the central tube 1129, accelerates the gases flow through the tube 1129, such that the gases exiting the tube 1129 are at a higher velocity than the surrounding gases flow through the prong 1105 outside of the tube 1129.

With reference to FIG. 10 an example configuration of a prong 1105 provided with multiple flow paths comprising a concentric flow path arrangement where a central tube 1129 extends along the longitudinal axis of the prong 1105 to separate the prong into inner and outer flow paths 1131, 1133. In the FIG. 10a example inner flow path 1131 is an inspiratory flow path, with the outer flow path 1133 being an expiratory flow path. In the FIG. 10b example the inner flow path 1131 is an expiratory flow path and the outer flow path 1133 is an inspiratory flow path.

The concentric nasal prongs 1105 may be sealing or non-sealing prongs.

Creating a seal with dual lumen nasal prongs 1105 will allow the expiratory flow to be conveyed through an expiratory conduit via a filter, to filter aerosols, or may be otherwise channeled to an external filter. If the prongs 1105 are not sealing, expiratory flow may also flow around the nasal prongs 1105 and directly to atmosphere.

Known filters which may be employed in the expiratory flow path include any one or more of HEPA filters, cyclonic filters, and electrostatic filters.

Where the prongs 1105 are configured such that the gases flow enters through the outer flow path 1133, which may be an outer annulus, this may reduce the incoming inspiratory gases flow velocity, as increasing the area of the outer annulus (with a larger diameter) relative to the central annulus results in a lower incoming gas velocity.

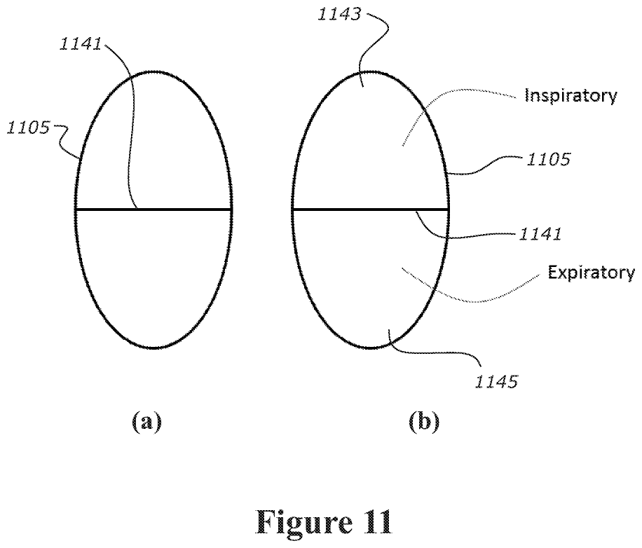

With reference to FIG. 11 another embodiment of a multiple flow path prong 1105 is provided in which the flow path through the prong 1105 is divided by a dividing wall 1141 extending across the flow path.

The dividing wall 1141 can be provided in any orientation within the prong 1105, but in the example shown extends across the prong 1105 at its widest region, so as to define an anterior flow path 1143 and a posterior flow path 1145. In alternative embodiments the dividing wall 1141 could be arranged so as to define two side by side flow paths.

The prong 1105 thus comprises two flow paths through the prong 1105, and as with the concentric embodiment described above, each prong 1105 can therefore comprise an inspiratory flow path and an expiratory flow path, or multiple inspiratory flow paths, or multiple expiratory flow paths.

The dual aperture nasal prongs may be sealing or non-sealing prongs.

Adult nares are typically substantially elliptical in shape, when viewed along their longitudinal axes. Each prong 1105 may also be elliptical when viewed along its longitudinal axis and is separated into two flow paths. For example, with reference to FIG. 11b, the anterior part 1143 of the ellipse is used for inspiratory gas, and the posterior part 1145, which could be optionally filtered, is used for expiratory flow.

Each prong 1105 may be any of the following shapes, when viewed along its longitudinal axis:
a. tear-drop, comprising a wider end tapering to a narrower opposed end;
b. kidney bean, comprising a semi-circular lower portion and two spaced apart protruding upper portions;
c. peanut, comprising two wider opposed ends, and a narrower waist portion intermediate the two ends;
d. circular.

The effective hydraulic diameter (and therefore flow resistance) of the inspiratory and expiratory flow paths could be different. A high resistance (smaller hydraulic diameter) inspiratory path will increase inspiratory jet velocity and CO2 clearance for a given flowrate, while a low resistance (larger hydraulic diameter) expiratory flow path will reduce work of breathing throughout the breath cycle. A smaller inspiratory diameter may lead to a reduction in aerosol generation as previously discussed. Resistance to flow is proportional to the equivalent hydraulic diameter to the 4th power, so small changes in hydraulic diameter will have a large effect on resistance.

Furthermore, the inspiratory and expiratory channels may be different lengths, and extend different lengths into the nares. It may be beneficial to have a longer expiratory channel so that the expiratory flow can enter the expiratory path before some aerosols can be shed from the nasal wall. The flow path lengths may be similar to those described above, namely between 4 and 30 mm. This can apply to the other above described prong embodiments too, for example, where a concentric central flow tube is provided.

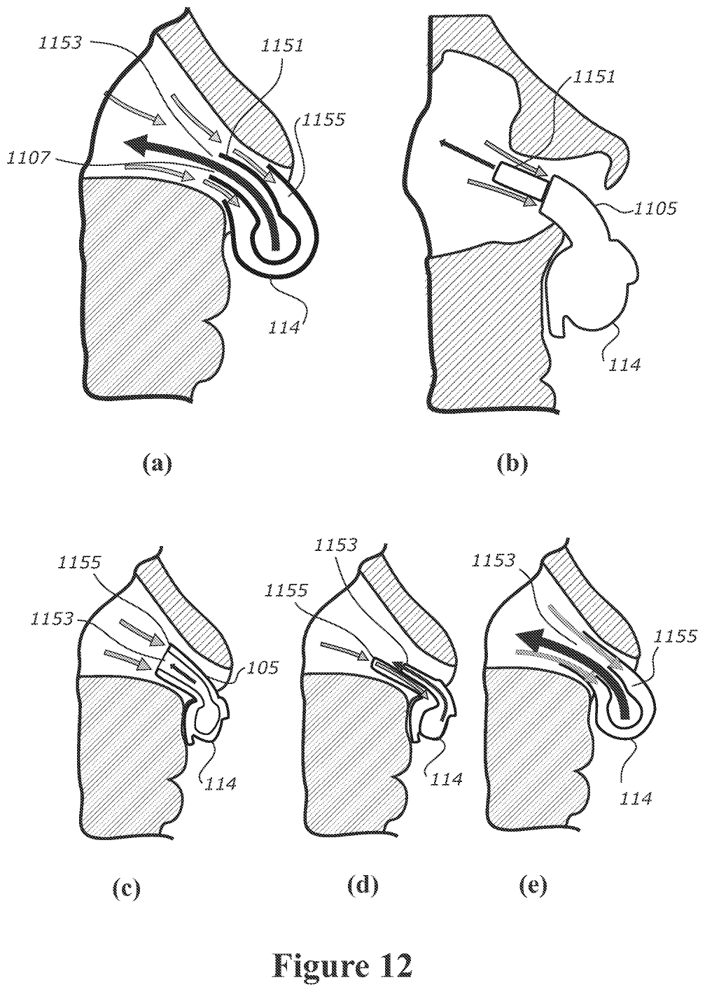

With reference to FIG. 12, various dual lumen prongs are shown, each prong 1105 comprising an inspiratory gases flow path, and a separate expiratory gases flow path. The expiratory gases flow path may deliver expiratory gases to the manifold 114, or the expiratory gases may be vented to atmosphere.

FIGS. 12a and 12b show an embodiment where the inspiratory gases flow path is through an innermost tube 1151, when viewed along the axis of the prong 1105. The prong 1105 may be considered to comprise an inspiratory gases channel 1153 and an expiratory gases channel 1155 defined internally and externally of the central flow tube 1151. In this example, the inspiratory gases channel 1153 projects further into the patient's nare than the expiratory gases channel 1155. In other words, the part of the prong 1105 that delivers inspiratory gases is longer (measured between the manifold 114 and the prong outlet opening 1107) than the part of the prong 1105 that removes expiratory gases.

With reference to FIG. 12c, the inspiratory and expiratory gases channels 1153, 1155 project the same distance into the patient's nare. In this example, the inspiratory gases channel 1153 is radially innermost. The inspiratory gases channel 1153 flares radially outwardly along its length, towards the outlet opening 1107.

With reference to FIG. 12d, in this example, the inspiratory and expiratory gases channels 1153, 1155 are adjacent, as per the example of FIG. 11. In this example, the expiratory channel 1155 projects further into the patient's nares than the inspiratory channel 1153, such that the expiratory channel inlet 1155A is further inside the patient's nose than the inspiratory channel outlet 1107. In this example, the inspiratory channel 1153 is adjacent, but external of the expiratory channel 1155, such that the channels 1153, 1155 are not concentric.

With reference to FIG. 12e, a concentric embodiment is shown, where the inspiratory and expiratory channels 1153, 1155 are concentric with the inspiratory channel 1153 being radially innermost. In this example the expiratory channel 1155 projects further into the patient's nare than the inspiratory channel 1153. In this example, the outlet of the inspiratory channel 1153 is contained inside the expiratory channel 1155, the inspiratory channel outlet 1107 being longitudinally spaced from the expiratory channel inlet 1155A.

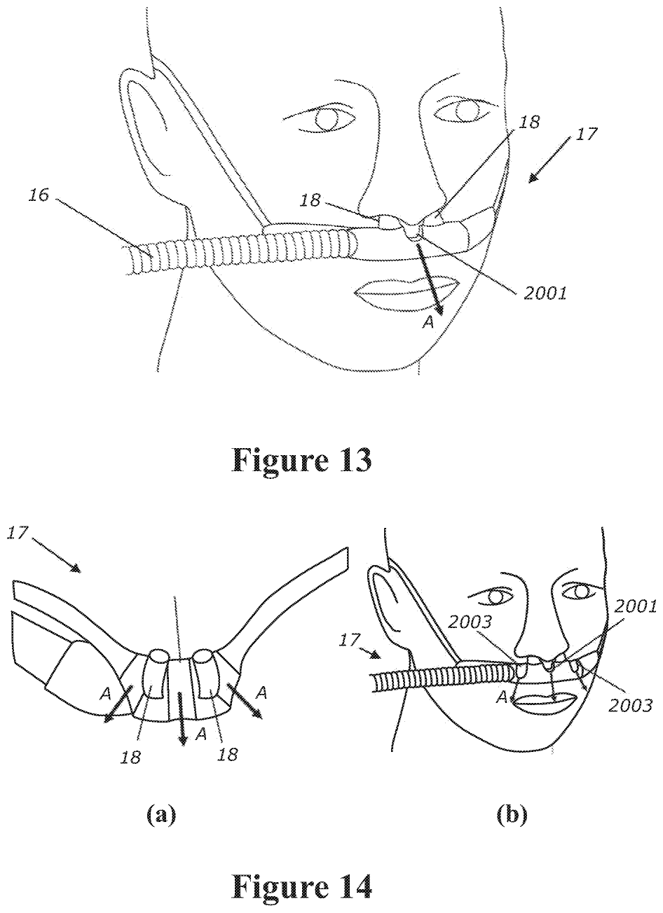

We refer now to FIGS. 13 to 20 in which a nasal cannula interface 17 is provided with one or more features configured to control or alter the direction and/or velocity of the gases flow into or out of the nasal cannula interface 17. As with the earlier described embodiments, a patient breathing conduit 16 is coupled at one end to the manifold 19 of the nasal cannula interface 17, a pair of nasal prongs 18 depending from the manifold 19.

Redirecting or controlling direction of gases exhaled by the patient can potentially reduce the risk of transmission of disease from potentially harmful aerosols expired by the patient. We have realised that exhaled gas that is exhaled in a generally forwards direction, i.e. forwardly away from the patients face, is more likely to come into contact with other persons near the patient, such as hospital staff, visitors or the like. We describe below a nasal cannula interface 17 where the exhaled gas can be redirected, such as downwards, upwards, sideways and/or behind the patient, so that the risk of others coming into contact with aerosols in the exhaled gas flow may be reduced. We also describe below a nasal cannula interface in which the exhaled gas flow has a reduced velocity and is unlikely to be able to carry aerosols as far, thus also reducing risk.

With reference to FIGS. 13 and 14 initially, a nasal cannula interface 17 in accordance with these figures comprises one or more cannula body features to divert exhaled gas flow. These features comprise structures or modifications to the cannula body, and in particular to the prongs 18 and/or manifold 19, that can encourage exhaled gases to travel outwardly away from the patient in more desirable directions, i.e. directions that may help to reduce risk of transmission of diseases. Such features may also reduce turbulence in the gases flow which may contribute to reducing dispersal of droplets/aerosols.

Nasal cannula interface 17 is provided with such a feature comprising a central channel 2001 located between the pair of prongs 18. The channel 2001 is elongate and extends generally forwardly and downwardly, away from the patient's face, towards the patient's mouth. The longitudinal axis of the channel 2001 is generally perpendicular to the longitudinal axis of the manifold 19. The longitudinal axis of the channel 2001 is downwardly directed, so as to be configured to direct expiratory flow in a substantially downwards direction. The lowermost point of the channel 2001 is below the base of the prongs 17, that is, below where the prongs 17 intersect the manifold 19.

Channel 2001 is angled such that flow exiting the nares that contacts the channel 2001 is directed generally downwards, as indicated by arrow A in FIG. 13.

Channel 2001 in this embodiment is located centrally in the manifold 19, between the two prongs 18.

Channel 2001 in this embodiment comprises a generally planar base surface, and opposed spaced apart generally planar side surfaces upstanding from the base surface.

In addition to the channel 2001, the base of the prongs 18 and/or the manifold region adjacent the base of the prongs 18 may have a generally downwardly sloping configuration to also direct exhaled gas downwards. In other words, one or more external surfaces of the prongs 18 and/or manifold 19 may be sloped downwardly, away from the face of the patient.

Referring to FIG. 14, in another embodiment, the manifold 19 may comprise additional channels being side channels 2003 located adjacent each prong 18, laterally spaced from the central channel 2001. Each prong 18 is between central channel 2001 and a respective side channel 2003.

Channels 2003 similarly direct gases flow in a controlled direction away from the patient. Channels 2003 may direct gases flow in a direction that is other than forwards.

In the embodiment shown, the side channels 2003 are configured such that exhaled gases flow coming into contact with the side channels 2003 is directed generally downwards and sidewards. The central channel 2001 directs expiratory flow from the medial side of each nare in a substantially downward direction. The side channels 2003 direct expiratory flow from the lateral side of each nare in a substantially downwards direction.

The central channel 2001 and/or one or both of side channels 2003 may be configured to direct expiratory flow at around a 45° angle downwards in one example. In other examples, the central channel may angled downwardly in the range of 5-85°, preferably in a range of 15-75°, more preferably in a range of 25-60°, and in some examples in a range of 40-50°.

Directing expiratory flow generally downwards encourages flow over the patients body/chest and/or patient bed if the patient is located in one. The directed expiratory gas flow may diffuse as it travels away from point of exhalation, which may assist to prevent or minimise turbulent airflow directly in front of the patients face. The central channel 2001 is therefore generally configured to direct any ‘concentrated’ aerosol laden gases out of the breathing zone of any caregivers in the near vicinity. After which any aerosols can diffuse out into the environment. The channel 2001 is configured to direct exhaled gases downwards, for example over the patient's body/chest and length of the bed, with the gases diffusing out as it goes (i.e. preventing or at least minimising turbulent airflow directly in front of the patients face).

The manifold 19 may have the central and side channels 2001, 2003 in combination or either of the central or side channels 2001, 2003.

Alternatively, instead of channels 2001, 2003, the manifold 19 may have one or more sloped regions to achieve similar effect. For example, the manifold 19 may have a sloped region between the prongs 18, extending generally down a front portion of the manifold 19; and/or sloped regions either side of the prongs 18.

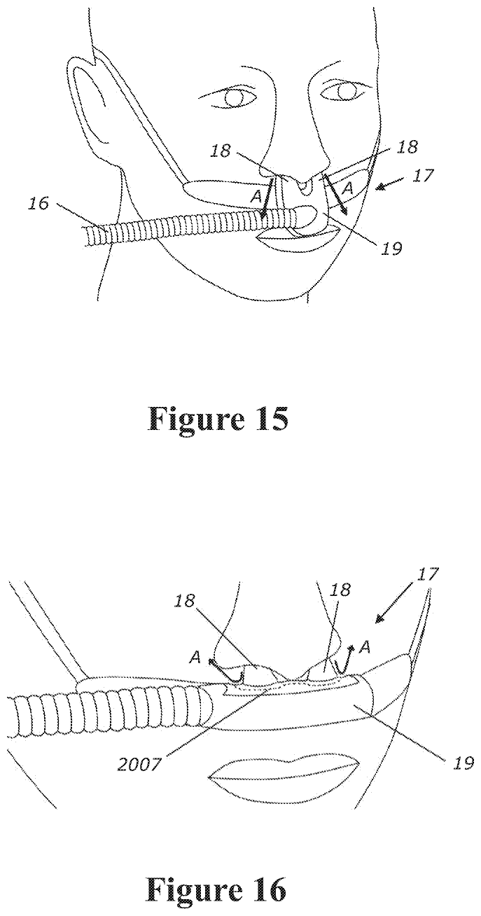

Referring to FIG. 15, in another embodiment, the overall shape of the manifold 19 can be narrowed such that the manifold 19 does not extend laterally beyond the outside of the prongs 18. This allows expiratory gases flow on the outside of the prongs 18 to travel downwards uninterrupted. This shape may also include the central channel 2001, as described above with reference to FIGS. 13 and 14.

The cannula may also have a reduced proximal-to-distal thickness, with a flat distal surface on the side of the manifold 19 facing away from the patient, to allow flow to travel substantially downwards without interruption.

In this embodiment, the manifold 19 is elongate. The longitudinal axis of the manifold 19 is substantially vertical, as compared to the manifold 19 in earlier embodiments where the longitudinal axis is substantially horizontal. The manifold 19 in this embodiment comprises a lower body portion 2005, to which breathing conduit 16 is connected, with the prongs 18 extending upwardly from the body portion 2005.

The relatively narrow manifold 19 in this embodiment provides expiratory gases flow to flow generally downwardly in the direction of arrows A in FIG. 15, without the gases flow being impeded by a surface of the manifold 19.

Referring to FIG. 16, in another embodiment, one or more depressions 2007 may be formed on an upper surface of the manifold 19, surrounding and adjacent to the base of the prongs 18. The one or more depressions 2007 are configured to direct flow exiting the nares in a generally upwards direction, as indicated by arrows A. The one or more depressions 2007 are configured such that expiratory gases enters the depressions 2007 from the nares, contact a base surface of the depressions 2007, and rebound from that base surface in a generally upward direction, away from the manifold 19. The depressions 2007 may be configured to direct the expiratory gases laterally outwardly as well as upwardly, away from the nares of the patient. The depressions and manner in which expiratory gases enter and rebounds from the base surface may assist to prevent, reduce or minimize turbulence and/or diffusion of the expiratory gases flow.

The one or more depressions 2007 may comprise a single elongate trough around both prongs, or may comprise a pair of troughs, each around a respective prong 18.

The or each depression 2007 comprises a sidewall that is inclined or curved such that gases flow enters the depression 2007 in a downward direction, and follows the curve or angle of the sidewall to exit the depression 2007 in a generally upward direction.

In some embodiments, the shape of the prongs 18 may become wider towards the manifold 19, forming a continuous surface with the sidewall of the depression 2007 to create a smooth flow profile.

The one or more depressions 2007 therefore provide a gases flow directing feature, on the manifold 19, to direct the gases flow from the nares in a desired direction. This can help to minimize gases flow in a generally forward direction.

The embodiments of FIGS. 13 to 16 comprise one or more gases flow directing features configured to:
d. direct gases flow from the patient's nares in one or more desired directions; and/or
e. prevent or reduce gases flow in one or more undesired directions.

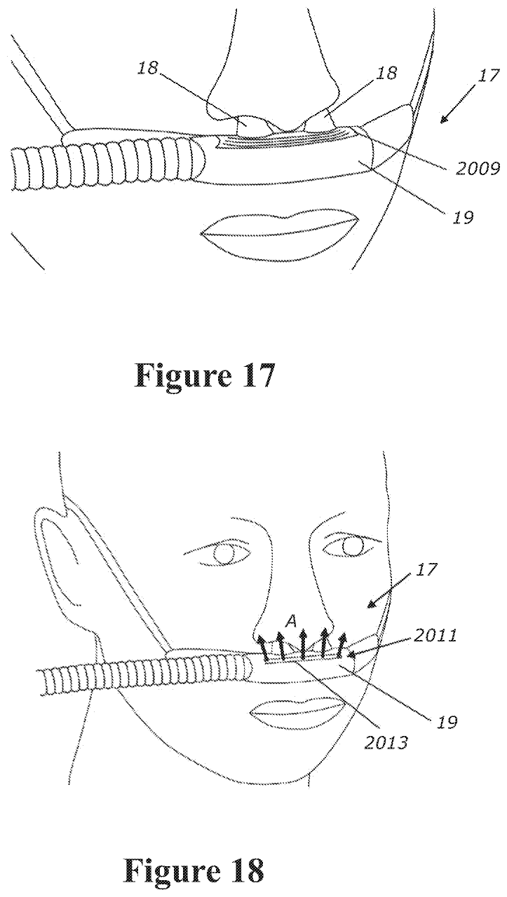

We refer now to FIG. 17 in which a cannula interface 17 is provided comprising one or more features configured to decrease the velocity of exhaled gases flow.

In this embodiment the cannula interface 17 may comprise a surface textured portion 2009, under the nares, that is configured to decrease gases flow velocity upon exhaled gases flow exiting the nares and contacting the surface textured portion.

The surface textured portion 2009 is preferably provided on an upper and/or front surface of the manifold 19. The surface textured portion 2009 is preferably provided adjacent the base of the prongs 18, for example adjacent and front of the base of the prongs 18, and/or to the side of either or both prongs 18, and/or inbetween the prongs 18.

The surface textured portion 2009 may comprise a recessed portion, and an adjacent raised portion. The base of the recessed portion and/or the peak of the raised portion, may comprise a substantially flat surface, an arcuate surface, or an intersection between two inclined or curved side surfaces.

The surface textured portion 2009 may comprise a plurality of recessed portions and/or a plurality of adjacent raised portions.

The recessed portion(s) may comprise an elongate channel. The raised portion(s) may comprise an elongate ridge. The elongate channel and/or the elongate ridge may be substantially straight, substantially arcuate, or may comprise one or more substantially straight portions and/or one or more substantially arcuate portions.

In the embodiment shown in FIG. 17, the surface textured portion 2009 comprises a plurality of arcuate channels and a plurality of arcuate adjacent ridges. The surface textured portion extends across the top surface of the manifold 19, in front of both prongs and in front of the space between the prongs 18, and curve around the laterally outermost part of each prong 18.

The surface textured portion 2009 may comprise one, or a series of, smooth ridges and depressions, sharper peaks and valleys, arrangement of circular dimples for example.

The surface textured portion 2009 can extend over a portion of the top of the manifold 19 or may extend over any region of the manifold 19 and/or prongs 18 onto which exhaled gases may flow. The surfaced texture portion 2009 may also have some effect on changing the direction of exhaled flow away from a generally forwards direction.

We refer now to FIGS. 18 to 20 in which embodiments are shown that comprise one or more features configured to divert exhaled flow/particles using gas flow.

In these embodiments, the manifold 19 is configured such that a portion of the gases flow delivered into the manifold 19 is used to generate an air curtain that may act to divert expiratory gases flow (and expiratory particles in that gases flow) exiting the nose and/or mouth of the patient. The generated air curtain can dilute the expiratory particles, and/or direct the gases flow to a more desirable location.

In these embodiments the nasal cannula 17 is provided with an additional outlet 2011 for the inspiratory gases flow, in addition to the outlet through each prong 18. Inspiratory gases flow enters the manifold 19 from the breathing conduit 16, and flows from the nasal cannula interface 17 via prongs 18. In these embodiments some of the inspiratory gases flow also flows from the nasal cannula interface 17 via the additional outlet 2011. The additional outlet 2011, which may comprise a single or a plurality of outlet ports 2013, is positioned such that the inspiratory gases flow through the additional outlet 2011 influences the direction of the expiratory gases flow from the patient.

The manifold 19 may therefore comprise an additional outlet 2011 comprising one or more outlet ports 2013 configured to allow a portion of delivered flow to exit the manifold 19 at a relatively high velocity. Flow to the patient is simultaneously delivered through the prongs 18 as per standard delivery of therapy.

The outlet port 2013 of the additional outlet 2011 may for example be elongate. The outlet port 2013 may be circular, elliptical, square or rectangular.

Referring to FIGS. 18 to 20, in one embodiment, an outlet port 2013 of the additional outlet 2011 may be in the form of a continuous, elongate slit.

The outlet port or ports 2013 can extend at least part way along the length of the manifold 19.

The outlet port, or ports, 2013 can be located at the top and/or front surface of the manifold 19, underneath the base of the prongs 18.

The outlet port, or ports, 2013 can be located at the front of the manifold 19, and spaced a predetermined distance away from the base of the prongs 18.

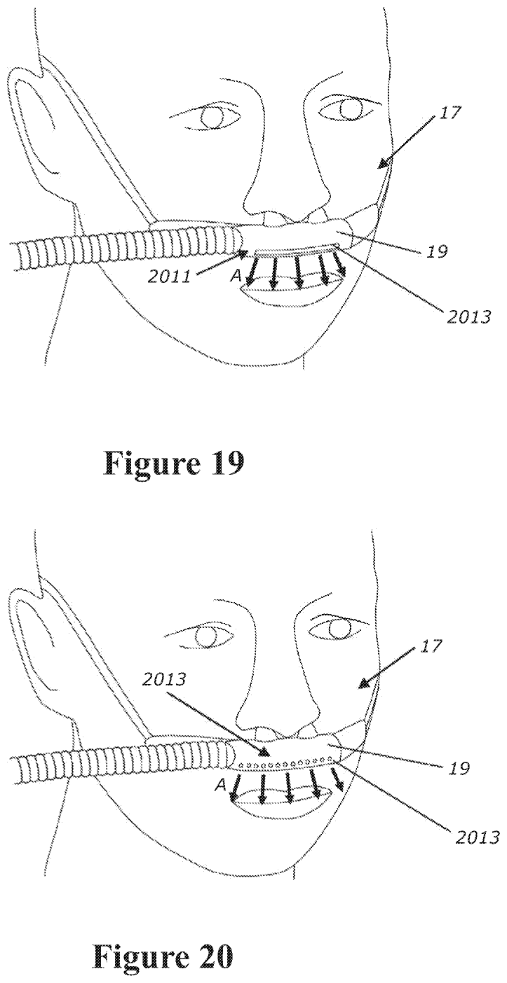

The outlet port, or ports, 2013 can be angled or otherwise configured such that flow exits the manifold 19 in a predetermined direction. For example, the outlet port, or ports, 2013 can be angled or otherwise configured such that a portion of the inspiratory gases flow exits the manifold body 19 in a generally upwards or downwards direction, for example as shown by arrows A in FIGS. 18 and 19 respectively. An upwards flow may assist to redirect at least some particles exhaled from the nose. A downwards flow may assist to redirect at least some particles exhaled or otherwise exiting from the mouth.

A plurality of outlet ports 2013 may be provided. For example, one outlet port 2013 could be positioned on an upper part of the manifold 19 to redirect particles exhaled from the nose, whilst another outlet port 2013 could be positioned on a lower part of the manifold 19 to redirect particles exhaled from the mouth.

With reference to FIG. 20, additionally or alternatively, the additional outlet 2011 may comprise an array of outlet ports 2013.

The array of outlet ports 2013 may be arranged on a front portion of the manifold 19. The outlet ports 2013 may be arranged in a line extending along at least a portion of the length of the manifold 19. The line may be a straight or an arcuate line.

The outlet ports 2013 may be uniformly spaced apart from each other. The outlet ports 2013 may be angled or otherwise configured such that flow exiting the outlet ports 2013 is directed in one, or a plurality of, directions. As per the embodiments of FIGS. 18 and 19, the outlet ports 2013 may direct flow upwards so as to redirect particles exhaled from the nose. In other embodiments, the outlet ports 2013 may direct flow downwards so as to redirect particles exhaled from the mouth.

The outlet ports 2013 may also be configured to direct the air curtain of inspiratory gases flow in a sideways direction, laterally outwardly of the prongs 18. In one example, where the outlet ports 2013 direct the flow in a generally downwards direction (for example as per FIG. 19), the outlet ports 2013 in more laterally distant positions may be directed slightly outwards from the centre, to provide an air curtain that covers a wider area.

The or each outlet port 2013 may be initially sealed with a closure or plug (not shown). The closure or plug can be removed in the event it is desired to create the air curtain by using a portion of the inspiratory gases flow, thus fully opening, or at least partially opening, the one or more outlet ports 2013. For example, if a plurality of outlet ports 2013 are provided, the closure or plug may be removed from all, some, or only one of these. The closure or plug could comprise a movable component of the nasal cannula interface, a separate but reusable component, or could comprise a disposable component. The closure may comprise an adhesive strip that can be placed over a desired number of the outlet ports 2013.

The size of the one or more outlet ports 2013 determines the velocity of inspiratory gases exiting through it. Smaller sized ports 2013 may allow for a faster velocity air curtain that more effectively diverts infectious particles. Larger ports 2013 may allow for a slower velocity air curtain that is more comfortable for the patient.

A plurality of outlet ports 2013 may be provided in which at least one outlet port is configured such that inspiratory gases pass through that outlet port at a relatively high velocity, whilst at least another outlet port 2013 is provided and configured such that inspiratory gases pass through that outlet port 2013 at relatively slow velocity. In one example one or more inner outlet ports 2013 may be configured for relatively high gases velocity, whilst one or more outer outlet ports 2013 may be configured for relatively slow gases velocities.

A plurality of outlet ports 2013 can be provided of different sizes. For example, one or more larger outlet ports 2013 may be outermost, and one or more smaller outlet ports 2013 may be innermost.

The relative sizes, and the number of outlet ports 2013 can be configured to achieve the desired effect, that is to divert infectious particles, and/or provide the desired level of comfort to the patient, and/or create the desired exit velocity profile for the gases.

The one or more outlet ports 2013 can comprise a combination of fast/slow or different sized ports. Arrangement of the sizes can be configured to achieve the desired effect, for example, one or more smaller sized (faster) on the inside; one or more larger (slower) on outer.

The effective size of the or each outlet port 2013 may be variable. For example, an adjustable valve or adjustable closure may be provided, to vary the effective size of the or each outlet port 2013. As noted above, the closure could comprise an adhesive strip providing an adjustable closure configured to selectively cover or open one or more of the outlet ports 2013. An adjustable closure may be provided configured such that, for example:
a. Only the inner most outlet ports 2013 are open;
b. Only the outermost outlet ports 2013 are open;
c. The right hand side outlet ports are open; and/or
d. The left hand side outlet ports 2013 are open.

The amount of delivered flow may be increased relative to a nasal cannula interface without the additional outlet, to account for the portion of the inspiratory gases flow that exits through the additional outlet and is not therefore delivered through the prongs 18 and used for therapy. This increase in flow may be generated by a faster blower speed, or from a separate inspiratory gases supply or device.

It is envisaged that features of any of the above described embodiments may be combined with features of any one or more of other described embodiments.

The various configurations described are exemplary configurations only.

Any one or more features from any of the configurations may be used in combination with any one or more features from any of the other configurations.

Reference to any prior art in this specification is not, and should not be taken as, an acknowledgement or any form of suggestion that the prior art forms part of the common general knowledge in the field of endeavor in any country in the world.

Where reference is used herein to directional terms such as ‘up’, ‘down’, ‘forward’, ‘rearward’, ‘horizontal’, ‘vertical’ etc., those terms refer to when the apparatus is in a typical in-use position, and are used to show and/or describe relative directions or orientations.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The terms “approximately,” “about,” and “substantially” as used herein represent an amount close to the stated amount that still performs a desired function or achieves a desired result. For example, in some embodiments, as the context may permit, the terms “approximately”, “about”, and “substantially” may refer to an amount that is within less than or equal to 10% of, within less than or equal to 5% of, and within less than or equal to 1% of the stated amount.

Reference to any prior art in this specification is not, and should not be taken as, an acknowledgement or any form of suggestion that that prior art forms part of the common general knowledge in the field of endeavour in any country in the world.

The disclosed apparatus and systems may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

Depending on the embodiment, certain acts, events, or functions of any of the algorithms, methods, or processes described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially.

It should be noted that various changes and modifications to the presently preferred embodiments described herein will be apparent to those skilled in the art. Such changes and modifications may be made without departing from the spirit and scope of the disclosed apparatus and systems and without diminishing its attendant advantages. For instance, various components may be repositioned as desired. It is therefore intended that such changes and modifications be included within the scope of the disclosed apparatus and systems. Moreover, not all of the features, aspects and advantages are necessarily required to practice the disclosed apparatus and systems. Accordingly, the scope of the disclosed apparatus and systems is intended to be defined only by the claims that follow.

What is claimed is:

1. A nasal cannula interface for a respiratory support system configured to receive a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient;
wherein the nasal prong defines a plurality of gas flow paths extending through the prong.

2. The nasal cannula interface of claim 1 wherein one of the flow paths is an inspiratory flow path, configured to deliver the breathable gases flow to the patient; and another one of the flow paths is an expiratory flow path, configured to receive expiratory gases flow from the patient.

3. The nasal cannula interface of claim 1 wherein a plurality of the gas flow paths are inspiratory flow paths.

4. The nasal cannula interface of claim 1 wherein a plurality of gas flow paths are expiratory flow paths.

5. The nasal cannula interface of any one of the preceding claims wherein the gases flow through one gas flow path is different from the gases flow through another gas flow path.

6. The nasal cannula interface of any one of the preceding claims wherein the gases flow through one gas flow path is at a higher flow velocity than the gases flow through another gas flow path.

7. The nasal cannula interface of any one of the preceding claims wherein the cross sectional area of one gases flow path is different from the cross sectional area of another gases flow path.

8. The nasal cannula interface of claim 7 wherein the cross sectional area of an expiratory flow path is greater than the cross sectional area of an inspiratory flow path.

9. The nasal cannula interface of any one of the preceding claims comprising concentric gas flow paths through the prong.

10. The nasal cannula interface of claim 9 wherein a flow path that is nearer a longitudinal axis of the prong is configured to transport a higher flow velocity of gases than a flow path that is further from the longitudinal axis of the prong.

11. The nasal cannula interface of claim 9 wherein both flow paths are expiratory flow paths.

12. The nasal cannula interface of claim 9 wherein a flow path that is nearer a longitudinal axis of the prong is an expiratory flow path, and a flow path that is further from the longitudinal axis of the prong is an inspiratory flow path.

13. The nasal cannula interface of claim 9 wherein a flow path that is nearer a longitudinal axis of the prong is an inspiratory flow path, and a flow path that is further from the longitudinal axis of the prong is an expiratory flow path.

14. The nasal cannula interface of any one of the preceding claims comprising adjacent flow paths through the prong.

15. The nasal cannula interface of claim 14 wherein the flow paths are defined by a dividing wall extending along the length of the prong.

16. The nasal cannula interface of claim 15 wherein the dividing wall divides the prong into flow paths of equal transverse cross section.

17. The nasal cannula interface of claim 15 or 16 comprising a plurality of dividing walls.

18. The nasal cannula interface of any one of the preceding claims wherein the hydraulic diameter and therefore flow resistance of the inspiratory and expiratory flow paths is different.

19. The nasal cannula interface of claim 18 wherein the hydraulic diameter of the inspiratory flow path is higher than that of the expiratory flow path.

20. The nasal cannula interface of claim 18 wherein flow resistance of the inspiratory flow path is lower than that of the expiratory flow path.

21. The nasal cannula interface of any one of claims 14 to 20 wherein the dividing wall divides the flow path into an anterior flow path and a posterior flow path.

22. The nasal cannula interface of any one of claims 14 to 21 wherein the prongs are elliptical, when viewed along their longitudinal axis.

23. A nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
wherein the flow path is configured such that a plug flow velocity profile is delivered from an outlet of the prong into the patient's nare(s).

24. The nasal cannula interface of claim 23 wherein the nasal prong comprises one or more flow guide formations or features in the flow path, to guide the gases flow along the flow path such that plug flow is delivered.

25. The nasal cannula interface of claim 24 wherein the flow guide formation comprises a flow vane.

26. A nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient;
wherein the nasal prong defines a flow path through the nasal prong between an inlet opening of the prong and an outlet opening of the prong, the cross sectional area of the flow path being greater at or adjacent the prong outlet opening than at or adjacent the prong inlet opening.

27. The nasal cannula interface of claim 26 wherein the prong is outwardly flared at the outlet opening.

28. The nasal cannula interface of claim 26 or claim 27 wherein the cross sectional area of the flow path increases along the length of the prong.

29. The nasal cannula interface of claim 28 wherein the cross sectional area increases over at least half the length of the prong.

30. The nasal cannula interface of any one of claims 26 to 29 wherein the length of the nasal prong is such that the nasal prong extends sufficiently into the nasal cavity of the nose of the patient such that the outlet of the nasal prong is beyond the nasal valve of the nose of the patient.

31. The nasal cannula interface of any one of claims 26 to 30 wherein the nasal prong is arcuate.

32. The nasal cannula interface of any one of claims 26 to 31 comprising a manifold, the nasal prong extending from the manifold, the manifold comprising a lip contacting portion, the nasal prong being arcuate, wherein the lip contacting portion of the manifold and the curve of the arcuate prong, are configured such that the prong engages part of the nares of the patient, whilst leaving a clearance between the remainder of the prong and the nares for an expiratory gases flow.

33. A nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
wherein the nasal prong has an external profile, the nasal prong being adjustable such that a physical characteristic of the nasal prong can be adjusted to alter the external profile of the nasal prong.

34. A nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
wherein the nasal prong comprises one or more flow altering surface features configured to alter the flow of gases along the nasal prong.

35. The nasal cannula interface of claim 34 wherein the flow altering surface feature is on an external surface of the prong.

36. The nasal cannula interface of claim 34 or 35 wherein the flow altering surface feature is on an internal surface of the prong.

37. The nasal cannula interface of any one of claims 34 to 36 comprising a plurality of flow altering surface features.

38. The nasal cannula interface of any one of claims 34 to 36 comprising one or more elongate grooves.

39. The nasal cannula interface of claim 38 wherein the one or more elongate grooves extend helically around at least part of the prong.

40. The nasal cannula interface of any one of claims 34 to 39 wherein the flow altering surface feature is configured to vary friction between the prong and the gases flow.

41. The nasal cannula interface of claim 34 wherein the flow altering surface comprises one or more dimples.

42. The nasal cannula interface of claim 34 wherein the flow altering surface comprises one or more projections.

43. The nasal cannula interface of claim 42 wherein the one or more projections comprise a rib or ridge or dimple.

44. The nasal cannula interface of any one of claims 34 to 43 wherein the prong comprises at least a portion of increased surface roughness.

45. The nasal cannula interface of claim 44 wherein the portion of increased surface roughness comprises a coating.

46. The nasal cannula interface of claim 44 wherein the exterior of the prong is textured or comprises a textured portion.

47. The nasal cannula interface of claim 44 wherein the interior of the prong is textured or comprises a textured portion.

48. The nasal cannula interface of any one of claims 34 to 47 comprising a tube inside the prong to divide the gases flow path through the prong into a plurality of flow paths, the tube being configured such the gases flow path through the tube conveys a portion of the gases flow at a higher velocity than the portion of the gases flow outside of the tube.

49. A nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient; the nasal prong defining at least one flow path through the prong;
wherein the flow path is configured such that the gases flow through the flow path comprises a higher velocity component, adjacent a longitudinal axis of the nasal prong, and a lower velocity component radially spaced from the longitudinal axis.

50. The nasal cannula interface of claim 49 comprising a tube inside the prong to divide the gases flow path through the prong into a plurality of flow paths, the tube being configured such the gases flow path through the tube conveys a portion of the gases flow at a higher velocity than the portion of the gases flow outside of the tube.

51. A nasal cannula interface for a respiratory support system configured to generate a breathable gases flow, the nasal cannula interface comprising:
a. an inlet to receive the gases flow;
b. at least one nasal prong configured to receive the gases flow from the inlet, and to be received in, and to deliver the gases flow to, a nare of the patient;
wherein the nasal prong defines a plurality of separate gas flow paths extending through the prong, one of the flow paths comprising an inspiratory flow path for delivering the gases flow to the patient's nare through an inspiratory outlet of the prong; another flow path comprising an expiratory flow path for receiving expiratory gases from the patient through an expiratory inlet of the prong; wherein the expiratory outlet extends further into the patient's nare than the inspiratory outlet.

52. The nasal cannula interface of claim 51 wherein the expiratory flow path is longer than the inspiratory flow path.

53. The nasal cannula interface of any one of the preceding claims comprising:
a. a nasal cannula interface;
b. a nasal pillows interface;
c. a combination interface, comprising a nasal cannula and a cushion configured to seal against the patient's face.

54. A respiratory support apparatus comprising the nasal cannula interface of any one of claims 1 to 53, the apparatus comprising a flow generator configured to generate the gases flow.

55. The apparatus of claim 54 being a high flow apparatus configured to generate a high gases flow.

56. The apparatus of claim 54 or 55 further comprising any one or more of:
a. a humidifier configured to humidify the gases flow;
b. an inspiratory conduit configured to be connected to the nasal cannula interface and to receive the gases flow from the flow generator or humidifier;
c. an expiratory conduit configured to be connected to the nasal cannula interface to receive expiratory gases from the nasal cannula interface;
d. a controller configured to control the flow generator and/or the humidifier;
e. a user interface configured to enable the patient to control one or more aspects of the system.

57. The apparatus of claim 56 wherein the inspiratory conduit comprises one or more heating elements.

58. A nasal cannula interface assembly comprising:
a. the nasal cannula interface of any one of claims 1 to 53;
b. headgear configured to be secured to the nasal cannula interface and to secure the nasal cannula interface to the patient's head;
c. an inlet conduit configured to be fluidly connected to the nasal cannula interface.
